SEQUENCE LISTING (52 sequences)

SEQ ID NO: 1 - DNA, 23 bp, Artificial Sequence
  Quantitative RT-PCR Primers
ggctctacgaccttagactgtca

SEQ ID NO: 2 - DNA, 23 bp, Artificial Sequence
  Quantitative RT-PCR Primers
tgctgtttcctggtcctaaaata

SEQ ID NO: 3 - DNA, 24 bp, Artificial Sequence
  Quantitative RT-PCR Primers
accagaagtaaaagtgatcccaga

SEQ ID NO: 4 - DNA, 24 bp, Artificial Sequence
  Quantitative RT-PCR Primers
tccaccaaaagatggagataattt

SEQ ID NO: 5 - DNA, 26 bp, Artificial Sequence
  Quantitative RT-PCR Primers
ggtgtcaccttcgactgggattatcc

SEQ ID NO: 6 - DNA, 26 bp, Artificial Sequence
  Quantitative RT-PCR Primers
cattctggctgacattgtggatggtc

SEQ ID NO: 7 - DNA, 20 bp, Artificial Sequence
  Quantitative RT-PCR Primers
cagaaggagagcagaagtcc

SEQ ID NO: 8 - DNA, 19 bp, Artificial Sequence
  Quantitative RT-PCR Primers
ctccagggcttcatcgtta

SEQ ID NO: 9 - DNA, 25 bp, Artificial Sequence
  Quantitative RT-PCR Primers
cgttctctgccagcaacattaccac

SEQ ID NO: 10 - DNA, 25 bp, Artificial Sequence
  Quantitative RT-PCR Primers
cttgttgggtccacagacatcatgg

SEQ ID NO: 11 - DNA, 25 bp, Artificial Sequence
  Quantitative RT-PCR Primers
gtctgtaaagcaccatgtccgggag

SEQ ID NO: 12 - DNA, 25 bp, Artificial Sequence
  Quantitative RT-PCR Primers
ccacaggctggctttctacttgcac

SEQ ID NO: 13 - DNA, 23 bp, Artificial Sequence
  Quantitative RT-PCR Primers
agactgacgtgatctggaagaac

SEQ ID NO: 14 - DNA, 23 bp, Artificial Sequence
  Quantitative RT-PCR Primers
acttgatggtggctgtacatttt

SEQ ID NO: 15 - DNA, 24 bp, Artificial Sequence
  Quantitative RT-PCR Primers
gccatcaagaggatgaaagagtat

SEQ ID NO: 16 - DNA, 24 bp, Artificial Sequence
  Quantitative RT-PCR Primers
gctcccaagagattaatgatgttt

SEQ ID NO: 17 - DNA, 23 bp, Artificial Sequence
  Quantitative RT-PCR Primers
aggtttctcagccaactacagtg

SEQ ID NO: 18 - DNA, 23 bp, Artificial Sequence
  Quantitative RT-PCR Primers
actgtgaagatgcagtgatctga

SEQ ID NO: 19 - DNA, 23 bp, Artificial Sequence
  Quantitative RT-PCR Primers
gcaggtttctctctacgctatga

SEQ ID NO: 20 - DNA, 23 bp, Artificial Sequence
  Quantitative RT-PCR Primers
atacttctctggaaaccctggag

SEQ ID NO: 21 - DNA, 23 bp, Artificial Sequence
  Quantitative RT-PCR Primers
aagatcctcatgaaagttggaca

SEQ ID NO: 22 - DNA, 23 bp, Artificial Sequence
  Quantitative RT-PCR Primers
tgaacttctcccatttgtaccag

SEQ ID NO: 23 - DNA, 23 bp, Artificial Sequence
  Quantitative RT-PCR Primers
aagatgggaagatacgaggaaag

SEQ ID NO: 24 - DNA, 23 bp, Artificial Sequence
  Quantitative RT-PCR Primers
caagattttcttctggtgtcctg

SEQ ID NO: 25 - DNA, 26 bp, Artificial Sequence
  Quantitative RT-PCR Primers
gaatgtatcccccacaatggctgtcg

SEQ ID NO: 26 - DNA, 24 bp, Artificial Sequence
  Quantitative RT-PCR Primers
gcacggagagtggtgagtacagta

SEQ ID NO: 27 - DNA, 25 bp, Artificial Sequence
  Quantitative RT-PCR Primers
ctgtgagaccaacatcaatgagtgc

SEQ ID NO: 28 - DNA, 25 bp, Artificial Sequence
  Quantitative RT-PCR Primers
gcagtcatccaggttgatctcacag

SEQ ID NO: 29 - DNA, 24 bp, Artificial Sequence
  Quantitative RT-PCR Primers
ccaacatgctacctgcccaccatc

SEQ ID NO: 30 - DNA, 25 bp, Artificial Sequence
  Quantitative RT-PCR Primers
gggcagtaaggaagtactcagagac

SEQ ID NO: 31 - DNA, 26 bp, Artificial Sequence
  Quantitative RT-PCR Primers
ctggtggcactctttgttaccttgac

SEQ ID NO: 32 - DNA, 23 bp, Artificial Sequence
  Quantitative RT-PCR Primers
ccagctcgtacttctgtgacacc

SEQ ID NO: 33 - DNA, 20 bp, Artificial Sequence
  Quantitative RT-PCR Primers
cagagtccagcattcaccac

SEQ ID NO: 34 - DNA, 20 bp, Artificial Sequence
  Quantitative RT-PCR Primers
aggaattgccacagctgaat

SEQ ID NO: 35 - DNA, 24 bp, Artificial Sequence
  Quantitative RT-PCR Primers
gtggatctcgagcttttcccttcc

SEQ ID NO: 36 - DNA, 20 bp, Artificial Sequence
  Quantitative RT-PCR Primers
ccgggggtctttggggattc

SEQ ID NO: 37 - DNA, 25 bp, Artificial Sequence
  Quantitative RT-PCR Primers
gcctggattctgtctcccatgactc

SEQ ID NO: 38 - DNA, 22 bp, Artificial Sequence
  Quantitative RT-PCR Primers
cagccaggggtttttggggatg

SEQ ID NO: 39 - DNA, 27 bp, Artificial Sequence
  Quantitative RT-PCR Primers
ggggtccatctcagttcagtctctaac

SEQ ID NO: 40 - DNA, 27 bp, Artificial Sequence
  Quantitative RT-PCR Primers
cttgagagggctgtaacttggagatcc

SEQ ID NO: 41 - DNA, 23 bp, Artificial Sequence
  Quantitative RT-PCR Primers
gagggccagaagcagtgttacac

SEQ ID NO: 42 - DNA, 26 bp, Artificial Sequence
  Quantitative RT-PCR Primers
ctggctaaaactggacagtgattgtg

SEQ ID NO: 43 - DNA, 24 bp, Artificial Sequence
  Quantitative RT-PCR Primers
gcaagcgactggcgcaacaaaatc

SEQ ID NO: 44 - DNA, 25 bp, Artificial Sequence
  Quantitative RT-PCR Primers
cggtacttgtagttgggatggtcgc

SEQ ID NO: 45 - DNA, 24 bp, Artificial Sequence
  Quantitative RT-PCR Primers
tccaggtgtggctgaagaatggtg

SEQ ID NO: 46 - DNA, 25 bp, Artificial Sequence
  Quantitative RT-PCR Primers
ctgtgaagtccctcttatgctcctg

SEQ ID NO: 47 - DNA, 25 bp, Artificial Sequence
  Quantitative RT-PCR Primers
gcatcgggaacaacttcatggatgg

SEQ ID NO: 48 - DNA, 27 bp, Artificial Sequence
  Quantitative RT-PCR Primers
ctctgcatacttgactcccacattgac

SEQ ID NO: 49 - DNA, 24 bp, Artificial Sequence
  Quantitative RT-PCR Primers
catcgtgggacgtcccagacatca

SEQ ID NO: 50 - DNA, 26 bp, Artificial Sequence
  Quantitative RT-PCR Primers
acagcctgaatagccacatacatggc

SEQ ID NO: 51 - DNA, 26 bp, Artificial Sequence
  Quantitative RT-PCR Primers
gtcgtggagtctactggtgtcttcac

SEQ ID NO: 52 - DNA, 28 bp, Artificial Sequence
  Quantitative RT-PCR Primers
gttgtcatatttctcgtggttcacaccc

(19) United States
(12) Patent Application Publication
(10) Pub. No.: US 2015/0368617 A1
(43) Pub. Date: Dec. 24, 2015

(54) METHOD FOR FORMING ENDOTHELIAL CELLS

(71) Applicant: Korea Advanced Institute of Science and Technology (KAIST), Daejeon (KR)

(72) Inventors: Gou Young KOH, Daejeon (KR); Hyung Joon Joo, Seoul (KR); Dong Kyu Choi, Yeongcheon-si (KR); Jin-Sung Park, Seoul (KR); Sung Woo Cho, Seoul (KR)

(73) Assignee: Korea Advanced Institute of Science and Technology (KAIST), Daejeon (KR)

(21) Appl. No.: 14/272,168

(22) Filed: May 7, 2014

Related U.S. Application Data
(63) Continuation-in-part of application No. PCT/IB2013/002502, filed on Aug. 7, 2013.
(60) Provisional application No. 61/680,391, filed on Aug. 7, 2012.

(51) Int. Cl. C12N 5/071; C12N 5/077

(52) U.S. Cl. CPC C12N 5/0691; C12N 5/069; C12N 5/0657; C12N 2501/999; C12N 2506/02

(57) ABSTRACT

The present application discloses a method of promoting differentiation and expansion of endothelial cells (EC) and cardiomyocytes from a pluripotent cell, which includes contacting the pluripotent cell with a Rho-associated protein kinase (ROCK) suppressor.

### BACKGROUND OF THE INVENTION

1. Field of the Invention

The present application relates to a method of promoting differentiation and expansion of endothelial cells and cardiomyocytes.

2. General Background and State of the Art:

Therapeutic neovascularization using ECs derived from ESC and iPSC holds great promise for patients with various kinds of ischemic diseases [1]. In order to gain therapeutic competence, the ECs need to be of ample quantity as well as high purity after differentiation and purification process in vitro. Many attempts were made during the last decade to achieve this goal: research groups led by Nishikawa and Yamashita have each progressively established efficient systems for promoting the differentiation, specification, and expansion of ECs using ESC- and iPSC-derived Flk1+ MPCs [1-6]; by adapting these culture systems, researchers have found that differentiation, specification, and expansion of ECs from Flk1+ MPCs can be promoted by activation of cAMP/protein kinase A signaling [7-9], Ras-ERK signaling [10], PI3-kinase/Akt/PKC signaling [11], angiopoietin-1/Tie2 signaling [12], and suppression of TGF-1 receptor kinase signaling [13,14], as well as implementation of a more favorable and adaptable growth medium or microenvironment [15,16]; last but not least, researchers have identified VEGF-A as a crucial and potent factor for differentiation of ECs—studies have shown that supplementation of VEGF-A along with utilization of a defined medium eliminates the need for feeder cell co-culture, thereby minimizing the level of cell contamination and further enhancing the purity of ECs [4,6,13].

Since supplemental VEGF-A has been found to be the most essential component for differentiation and maintenance of ECs in a feeder cell-free microenvironment, we have investigated how VEGF-A regulates the differentiation of ECs. During the dissection of VEGF-A/VEGFR2 (KDR/Flk1) downstream signaling that underlies the VEGF-A-induced differentiation and expansion of ECs [17,18], we noticed that a small chemical compound Y27632, an inhibitor of Rho-associated protein kinase (ROCK), profoundly promotes the differentiation and expansion of ECs. ROCK is a major downstream effector protein of RhoA, which is one of Rho family small GTPases that control a diverse array of cellular processes including cytoskeletal dynamics, cell polarity, membrane transport, and gene expression[19-21]. ROCK consists of two subtypes, ROCK1 and ROCK2, which both carry critical functions in the regulation of actin cytoskeleton assembly across many cell types through direct activation of myosin light chain and inactivation of myosin phosphatase [19-22]. ROCK also plays a key role in myogenic differentiation [23-25], and recent studies have shown that Y27632 blocks the dissociation-induced apoptosis in human ESC through suppression of Rho-ROCK pathway-mediated hyperactivation of actomyosin [26-28].

In the present study, we demonstrate how suppression of ROCK profoundly promotes the differentiation and expansion of ECs in our established feeder cell-free, 2-dimensional (2D) Matrigel system. Moreover, we show how ROCK suppression could be applied in obtaining ECs from ESC or iPSC at a sufficiently high quantity and purity suitable for therapeutic purpose.

Once cardiomyocytes (CMs) are dead by severe damage such as acute coronary blockage or prolonged congestive heart failure, they do not have any regenerative capacity. Consequently, the heart that has dead CMs in a certain area loses structural and functional capacity as a blood pumping organ to the whole body. Replacement of dead CMs with fresh and healthy cardiac stem cell by implantation is the only solution for cure of such heart diseases. Therefore, cardiac stem cell therapy is a promising field in regenerative medicine. Currently many experiments and clinical trials using putative cardiac stem cells derived from adult and embryonic stem cells are investigated [29-31]. However, it is difficult to obtain sufficient number of cardiac stem cells using currently available techniques and methodologies.

Pluripotent stem cells (PSCs) such as embryonic stem cells (ESCs) and induced pluripotent stem cells (iPSCs) have become available resources for cardiac stem cells. Especially, Flk1+ mesodermal precursor cells (MPCs) from differentiated ESCs are identified as cardiovascular progenitor cells [4,32,33]. Flk1+ MPCs can give rise to CMs, endothelial cells (ECs), hematopoietic cells (HCs) and mural cells (MCs). Most studies for CMs differentiation reported that four major signaling pathways which are BMP, TGFB/activin/NODAL, WNT and FGF are involved in early cardiac differentiation [34]. For example, Dickkopf-related protein-1 (DKK-1) which is WNT inhibitor and NOGGIN which is BMP4 inhibitor are widely used for CMs differentiation from PSCs [34]. And most studies regarding CMs differentiation have been using embryoid body (EB) method. However, EB method is impossible to prospectively identify the cells that differentiate into CMs and is difficult to trace and analyze the cellular and molecular process of differentiation especially at the single cell level [35]. Also, EB method is difficult to sort out and obtain purified CMs.

Research group led by Yamashita reported a novel CM induction method in 2-dimensional, single cell-based manner from ESC-derived Flk1+ MPCs using OP9 feeder cell co-culture system [35]. They also reported that Cyclosporine A (CsA) which is immunosuppressant widely used in clinical settings, increased cardiac progenitors and CMs from mouse ESCs, iPSCs and human iPSCs-derived Flk1+ MPCs [36,37].

In the present study, we demonstrated that a small chemical compound Y27632, an inhibitor of ROCK, significantly and markedly promotes the differentiation and expansion of CMs with CsA in OP9 co-culture system. In addition, we found that Y27632 increased the number of CMs also in feeder-free condition.

### SUMMARY OF THE INVENTION

In one aspect, the present invention is directed to a method of promoting differentiation and expansion of endothelial cells (EC) from a pluripotent cell comprising contacting the pluripotent cell with Rho-associated protein kinase (ROCK) suppressor. The ROCK suppressor may be RKI, RKI-II, siRNA-ROCK1, si-RNA-ROCK2 or chemical compound specific to ROCK1 or ROCK2. In particular, the chemical compound specific to ROCK1 or ROCK2 may be Y27632. The method may further include contacting the pluripotent cell with VEGF-A. The pluripotent cell may be embryonic stem cell (ESC) or mesodermal stem cell (MSC). Further, the pluripotent cell may be a mammalian cell. And the mammalian cell may be mouse or human cell.

In another aspect, the present invention includes a method of promoting differentiation and expansion of cardiomyocytes (CM) from a pluripotent cell comprising contacting the pluripotent cell with Rho-associated protein kinase (ROCK) suppressor. The ROCK suppressor may be RKI, RKI-II, siRNA-ROCK1, si-RNA-ROCK2 or chemical compound specific to ROCK1 or ROCK2. In particular, the chemical compound specific to ROCK1 or ROCK2 may be Y27632. The method may further include contacting the pluripotent cell with an immunosuppressant, such as Cyclosporine A. The pluripotent cell may be embryonic stem cell (ESC) or mesodermal stem cell (MSC). Further, the pluripotent cell may be a mammalian cell. And the mammalian cell may be mouse or human cell.

In yet another aspect, the invention is directed to a method of creating new blood vessels comprising contacting pluripotent cell with Rho-associated protein kinase (ROCK) suppressor. The ROCK suppressor may be RKI, RKI-II, siRNA-ROCK1, si-RNA-ROCK2 or chemical compound specific to ROCK1 or ROCK2. In particular, the chemical compound specific to ROCK1 or ROCK2 may be Y27632. The method may further include contacting the pluripotent cell with VEGF-A. The pluripotent cell may be embryonic stem cell (ESC) or mesodermal stem cell (MSC). Further, the pluripotent cell may be a mammalian cell. And the mammalian cell may be mouse or human cell.

In still another aspect, the present invention is directed to a method of regenerating or healing a part of a heart organ comprising contacting pluripotent cell with Rho-associated protein kinase (ROCK) suppressor thereby creating endothelial cells and cardiomyocytes, and administering the obtained cells to the heart. The ROCK suppressor may be RKI, RKI-II, siRNA-ROCK1, si-RNA-ROCK2 or chemical compound specific to ROCK1 or ROCK2. In particular, the chemical compound specific to ROCK1 or ROCK2 may be Y27632. The method may further include contacting the pluripotent cell with VEGF-A or an immunosuppressant, such as Cyclosporine A. The pluripotent cell may be embryonic stem cell (ESC) or mesodermal stem cell (MSC). Further, the pluripotent cell may be a mammalian cell. And the mammalian cell may be mouse or human cell.

In another aspect of the invention, the present invention is directed to a method of regenerating or healing a part of a heart organ comprising administering pluripotent cells to the heart and contacting the pluripotent cells with Rho-associated protein kinase (ROCK) suppressor thereby generating endothelial cells and cardiomyocytes. The ROCK suppressor may be RKI, RKI-II, siRNA-ROCK1, si-RNA-ROCK2 or chemical compound specific to ROCK1 or ROCK2. In particular, the chemical compound specific to ROCK1 or ROCK2 may be Y27632. The method may further include contacting the pluripotent cell with VEGF-A or an immunosuppressant, such as Cyclosporine A. The pluripotent cell may be embryonic stem cell (ESC) or mesodermal stem cell (MSC). Further, the pluripotent cell may be a mammalian cell. And the mammalian cell may be mouse or human cell.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1D. ROCK suppression promotes the differentiation and expansion of ECs from Flk1+ MPCs. (FIG. 1A) Protocol for differentiation of ECs. ESCs were cultured without LIF for 4.5 days for mesodermal induction. Flk1+ MPCs were then sorted by MACS and cultured with VEGF-A (50 ng/ml) for 2 days otherwise indicated in the presence of each indicated inhibitor, and their differentiation status was analyzed on day 6.5. (FIG. 1B) Immunofluorescence image showing CD144+ EC colonies, ┌ SMA+MCs, and DAPI+ nuclei in differentiating Flk1+ MPCs. Scale bar represents 100┌ m. (FIG. 1C) Representative FACS analysis of CD31+/CD144+ ECs incubated with or without VEGF-A (50 ng/ml). (FIG. 1D) Relative population of CD31+/CD144+ ECs grown with different inhibitors. Population of CD31+/CD144+ ECs grown with only VEGF-A (Control) was regarded as 100%. Each group, n=3. *p<0.01 versus Control.

FIGS. 2A-2F. ROCK suppression promotes the differentiation and expansion of ECs from Flk1+ MPCs. (FIG. 2A) Representative FACS analysis of CD31+/CD144+ ECs incubated with PBS (Control) or Y27632 (10┌ M). (FIG. 2B) % of CD31+/CD144+ EC population incubated with various concentrations of Y27632. Each group, n=3. *p<0.01 versus 0. (FIG. 2C) Relative cell number of CD31+/CD144+ ECs incubated with various concentrations of Y27632. Each group, n=3. *p<0.01 versus 0. (FIG. 2D) Immunofluorescence images showing CD144+ EC colonies, ┌ SMA+MCs, and DAPI+ nuclei in differentiating Flk1+ MPCs incubated with PBS (Control) or Y27632 (10┌ M). Scale bars represent 100┌ m. (FIG. 2E and FIG. 2F) Comparisons of EC colony formation in a given area (cm²) and cell number in each EC colony at 36 hrs after the endothelial induction. Each group, n=4. *p<0.01 versus Control.

FIGS. 3A-3D. ROCK suppression promotes the differentiation and expansion of ECs from Flk1+ MPCs. (FIG. 3A) % of CD31+/CD144+ EC population incubated with PBS (Control) or Y27632 (10┌ M) in Flk1+ MPCs derived from different kinds of ESCs (E14tg2a and R1) and iPSC. Each group, n=3. *p<0.01 versus Control. (FIG. 3B) % of CD31+/CD144+ EC population incubated with various ROCK inhibitors. Each group, n=3. *p<0.01 versus Control. (FIG. 3C) Immunoblotting for RhoA, ROCK1, and ROCK2 in different cell types: ESC (E14tg2a), Flk1+ MPC, EC and MC. (FIG. 3D) % of CD31+/CD144+ EC population incubated with non-specific control siRNA (Control), siRNA for ROCK1 (siROCK1), ROCK2 (siROCK2), or both (siROCK1&2). Each group, n=4. *p<0.01 versus Control.

FIGS. 4A-4E. Expansion of differentiated ECs by ROCK suppression is mainly due to the promotion of EC proliferation and Flk1+ MPCs adhesion. (FIG. 4A) Diagram of time points (6, 12 and 36 hrs) for assays of proliferation, cell cycle, apoptosis and adhesion in differentiating Flk1+ MPCs incubated with VEGF-A (50 ng/ml) (“Control”) or VEGF-A (50 ng/ml) plus Y27632 (10┌ M) (“Y72632”). (FIG. 4B) Immunofluorescence images showing PHH3+/CD144+ proliferative EC colonies, and DAPI+ nuclei at 36 hrs. Scale bars represent 100 ┌ m. (FIG. 4C) Number of PHH3+/CD144+ ECs in cultured area (cm²) at 36 hrs. Each group, n=5. *p<0.05 versus Control. (FIG. 4D) Representative FACS analysis showing cell cycle in CD144+ ECs from differentiating Flk1+ MPCs at 36 hrs. (FIG. 4E) % of CD144+ ECs under each phase of cell cycle at 36 hrs. Each group, n=3. *p<0.01 versus Control.

FIGS. 5A-5C. Effect of ROCK suppression on apoptosis in the Flk1+ MPCs. (FIG. 5A) Representative FACS analysis showing Annexin V+/PI− apoptotic cells. (FIG. 5B) % of Annexin V+/PI− apoptotic cells at 6 and 36 hrs. Each group, n=4. n.s. represents statistically non-significant. (FIG. 5C) % of adherent cells onto plate at 6 hrs. Each group, n=5. *p<0.05 versus Control.

FIGS. 6A-6C. ROCK suppression increases BrdU+/CD144+ EC population derived from Flk1+ MPCs. (FIG. 6A) Diagram of time points (12 hrs and 36 hrs) for cell proliferation assay in differentiating Flk1+ MPCs incubated with VEGF-A (“Control”) or VEGF-A plus Y27632 (10┌ M) (“Y72632”). (FIG. 6B) Representative FACS analysis of BrdU+/CD144+ proliferative ECs in differentiating Flk1+ MPCs. (FIG. 6C) % of BrdU+/CD144+ EC population at 12 hrs and 36 hrs. Each group, n=3. *p<0.01 versus Control.

FIGS. 7A-7C. ROCK suppression promotes the differentiation and expansion of ECs from Flk1+ MPCs. (FIG. 7A) Division of group according to procedure for induction of ECs in the presence of VEGF-A and/or Y27632 (10┌ M) with or without removal of non-adherent cells at 6 hrs after plating. (FIG. 7B) Representative FACS analysis of CD31+/CD144+ ECs. (FIG. 7C) % of CD31+/CD144+ ECs. Each group, n=4. *p<0.01 versus Group 1; ^{#}p<0.01 versus Group 3.

FIGS. 8A-8C. Effect of VEGF-A in ROCK suppression-induced expansion of ECs. Flk1+ MPCs were incubated with indicated agents for 2 days, and analyses were performed on day 6.5. (FIG. 8A) Representative FACS analysis showing CD144+ ECs or ┌ SMA+MCs incubated with PBS (Control) or Y27632 (10┌ M) in the absence or presence of VEGF-A (50 ng/ml) and VEGF-Trap (5┌ g/ml). (FIG. 8B and FIG. 8C) % of CD31+/CD144+ EC population and ┌ SMA+MC population. Each group, n=3. *p<0.01 versus Control. Each group, n=4. *p<0.01 versus each Control.

FIG. 9. Schematic diagram depicting the negative involvement of ROCK in VEGF-A/VEGFR2/PI3-kinase/AKT signaling pathway via PTEN in differentiation of ECs, and the positive involvement of ROCK in differentiation of MCs via MLC in the Flk1+ MPCs. Specific inhibitors for each signaling molecules are indicated as red characters.

FIGS. 10A-10B. PI3-kinase/Akt signaling and MyPT-MLC signaling are involved in ROCK suppression-induced expansion of ECs. Flk1+ MPCs were incubated with indicated agents for 2 days, and analyses were performed on day 6.5. (FIG. 10A) Immunoblotting for pPTEN, PTEN, pAkt, Akt, pERK, ERK, pMyPT, MyPT, pMLC, MLC, ┌ SMA and GAPDH in Flk1+ MPCs treated with PBS (Control), Y27632 (10┌ M), VEGF-A (50 ng/ml; VA), or VA plus Y27632. The Flk1+ MPCs were cultured for 12 hrs, starved for 6 hrs with or without Y27632, and then treated with VEGF-A (50 ng/ml) for 10 min. Results from four independent trials were similar to each other. (FIG. 10B) Quantitative analyses of phosphorylations of AKT, PTEN, MyPT and MLC. Signal intensities were quantitatively evaluated using Image J software. Intensity of phosphorylated/total form of each protein in Control is regarded as 1 and relative intensities are expressed as fold. The mean of relative values from four independent experiments are represented. *p<0.01 versus Control. ^{#}p<0.01 versus VEGF-A. N.D represents non-detected.

FIGS. 11A-11D. ROCK suppression promotes the differentiation of ECs through PTEN-Akt signaling and inhibits the differentiation of MCs through MyPT-MLC signaling. (FIG. 11A) Representative FACS analysis of CD31+/CD144+ ECs incubated with 0.1% DMSO or LY294002 (20┌ M; LY) in the presence of PBS (Control) or Y27632 (10┌ M; Y). (FIG. 11B) % of CD31+/CD144+ EC population. Each group, n=3. *p<0.01 versus 0. Each group, n=3. *p<0.01 versus DMSO; ^{#}p<0.01 versus Y27632. (FIG. 11C) Representative FACS analysis of ┌ SMA+MCs or CD144+ ECs incubated with 0.1% DMSO or ML7 (5 ┌ M) in the presence of PBS (Control) or Y27632 (10┌ M). (FIG. 11D) % of ┌ SMA+MC population. Each group, n=3. *p<0.01 versus 0. Each group, n=3. *p<0.01 versus DMSO. n.s. represents statistically non-significant.

FIGS. 12A to 12B-6. ROCK suppression-induced ECs express EC-specific genes. (FIG. 12A) Diagram of cell preparations for phenotype analyses of EC and MC differentiation. Flk1+ MPCs were incubated with only VEGF-A (50 ng/ml) (Control) and VEGF-A (50 ng/ml) plus Y27632 (10┌ M) (Y72632) for 2 days. (FIG. 12B-1 to FIG. 12B-6) Representative FACS analysis for phenotypic markers in CD144+ cells; pan-endothelial, mural, and arterial-, venous- and lymphatic EC.

FIGS. 13A-13B. ROCK suppression-induced ECs express EC-specific genes. (FIG. 13A) Diagram of cell preparations for analyses of gene expression related to differentiation of ECs and MCs. The cells in diagram (FIG. 13A) were sorted with anti-ICAM2 antibody, and gene expressions in each population of ICAM2+ ECs were analyzed by quantitative RT-PCR. (FIG. 13B) Comparisons of EC and MC differentiation-related gene expressions. Each group, n=3. *p<0.01 versus each Control.

FIGS. 14A-14G. ROCK suppression continuously promotes the expansion of ECs. (FIG. 14A) ICAM2+ ECs were further incubated with PBS (Control), Y27632 (10┌ M), VEGF-A (50 ng/ml; VA) or Y27632 (10┌ M) plus VEGF-A (50 ng/ml) for 2 days, and analyses were performed at day 8.5. (FIG. 14B) Immunofluorescence images showing CD144+ EC colonies, ┌ SMA+MCs, and DAPI+ nuclei. Scale bars represent 100┌ m. (FIG. 14C) Representative FACS analysis of CD31+/CD144+ ECs. (FIG. 14D) % of CD31+/CD144+ EC population. Each group, n=3. *p<0.01 versus Control. (FIG. 14E) Relative cell number of ICAM2+ ECs. Each group, n=3. *p<0.01 versus Control; ^{#}p<0.01 versus VEGF-A. (FIG. 14F) Relative population of CD31+/CD144+ ECs. Population of CD31+/CD144+ ECs grown without VEGF-A and Y27632 was regarded as 100%. Each group, n=3. *p<0.01 versus Control; ^{#}p<0.01 versus VEGF-A. (FIG. 14G) % of adherent ICAM2+ECs onto plate at 6 hrs. Each group, n=3. *p<0.05 versus VEGF-A.

FIGS. 15A-15D. ROCK suppression continuously promotes the expansion of ECs. ICAM2+ ECs were further incubated with PBS (Control), Y27632 (10┌ M), VEGF-A (50 ng/ml; VA) or Y27632 (10┌ M) plus VEGF-A (50 ng/ml) for 2 days, and analyses were performed at day 8.5. (FIG. 15A) Representative FACS analysis showing cell cycle in ICAM2+ ECs at 12 hrs. (FIG. 15B) % of ICAM2+ ECs under each phase of cell cycle at 12 hrs. Each group, n=3. *p<0.01 versus Control; ^{#}p<0.01 versus VEGF-A. (FIG. 15C) Representative FACS analysis of Annexin V+/PI− apoptotic ECs. (FIG. 15D) % of Annexin V+/PI− ECs. Each group, n=3. *p<0.01 versus Control.

FIG. 16. Estimation and comparison of number of ECs generated from one ESC grown with indicated agents in the 2D Matrigel system at day 6.5 and 8.5. Estimation was calculated from 5 independent incubations.

FIGS. 17A-17E. VYI-ECs have neovasculogenic capabilities in vitro. (FIG. 17A) Diagram of VYI-EC preparation for analyses of neovasculogenic activities. VYI-ECs were applied for tube forming assay and scratch wound healing assay using VEGF-A (50 ng/ml) (“Control”) or VEGF-A (50 ng/ml) plus Y27632 (10┌ M) (“Y72632”) (FIG. 17B) Representative phase contrast images showing network and branch formations of VYI-ECs at 12 hrs. Scale bars represent 100┌ m. (FIG. 17C) Number of branch points in a given area (cm²). Branch point was defined as the contact point of three or more endothelial tubes. Each group, n=3. *p<0.05 versus Control. (FIG. 17D) Representative phase contrast images showing invasion of VYI-ECs (white dotted lines) into wound regions at 6 hrs. White solid lines indicate the boundaries of wounding. Scale bars represent 100┌ m. (FIG. 17E) % of invasions. Area of wound at 0 hr is regarded as 100%. Each group, n=3. *p<0.05 versus Control.

FIG. 18. VYI-ECs have neovasculogenic capabilities in microfluidic angiogenesis assay system. VYI-ECs were applied for microfluidic angiogenesis assay using VEGF-A (50 ng/ml) (“Control”) or VEGF-A (50 ng/ml) plus Y27632 (10┌ M) (“Y72632”). Left, representative phase contrast and immunofluorescence images showing the sprouting of CD144+/FITC-lectin+ ECs into ECM scaffold at 48 hrs after cell seeding. Upper ECM scaffold provides a negative gradient of Y27632 (0 to 10┌ M) (“Control”), whereas lower ECM scaffold provides a constant concentration of Y27632 (10┌ M) (“Y27632”). Nuclei were stained with DAPI. Scale bars represent 100┌ m. Right, average length of sprouting ECs. *p<0.05 versus Control.

FIG. 19. VYI-ECs have neovasculogenic capabilities in vivo. Diagram of in vivo Matrigel plug assay. VYI-ECs derived from iPSCs were mixed with Matrigel supplemented with VEGF-A (500 ng/ml), and implanted into the dorsal flank of Tie2-GFP mouse. Implanted Matrigel was immunostained for CD31+ blood vessels and stained for DAPI+ nuclei. Donor-derived CD31+/GFP− blood vessels are formed in the gel, while invasion of recipient-derived CD31+/GFP+ blood vessels into the gel is detected. White arrows indicate the implanted VYI-ECs that have been integrated into recipient vessel. Scale bars represent 50┌ m.

FIGS. 20A-20F. CsA promotes the differentiation and expansion of CMs from Flk1+ MPCs. (FIG. 20A) Protocol for differentiation of CMs. ESCs were cultured without LIF for 4.5 days for mesodermal induction. Flk1+ MPCs were sorted by MACS and co-cultured with OP9 cells, and their differentiation status was analyzed on day 10.5. (FIG. 20B) Live cell image showing ┌ MHC-GFP+CMs in differentiating Flk1+ MPCs. Scale bar represents 100 ┌ m. (FIG. 20C) Relative total cell number incubated with various concentrations of CsA. Population of total cells without CsA was regarded as 100%. Each group, n=3. (FIG. 20D) % of cTnT+ CM population incubated with various concentrations of CsA. Each group, n=3. *p<0.01 versus 0. (FIG. 20E) Representative FACS analysis of cTnT+ CMs incubated with or without CsA (2 μg/mL). (FIG. 20F) % of cTnT+CM population incubated with or without CsA (2┌ g/mL). Each group, n=4. *p<0.01 versus Control.

FIGS. 21A-21D. CsA promotes the differentiation and expansion of CMs from Flk1+ MPCs. (FIG. 21A) Live cell images showing ┌ MHC-GFP+CMs in differentiating Flk1+ MPCs incubated with or without CsA (2┌ g/mL). Scale bars represent 100┌ m. (FIG. 21B) Immunofluorescence images showing cTnT+CM and DAPI+ nuclei in differentiating Flk1+ MPCs incubated with or without CsA (2┌ g/mL). Scale bars represent 100┌ m. (FIG. 21C) % of ┌ MHC-GFP+ area in live cell image incubated with or without CsA (2┌ g/mL). n=3. *p<0.01 versus Control. (FIG. 21D) % of cTnT+ area in immunofluorescence image with or without CsA (2┌ g/mL). n=3. *p<0.01 versus Control.

FIGS. 22A-22C. Combination of ROCK suppression and CsA additively promotes the differentiation and expansion of CMs from Flk1+ MPCs. (FIG. 22A) Diagram of cell preparations for analyses of CM differentiation. Flk1+ MPCs were co-cultured with OP9 cells and incubated with CsA (2┌ g/mL) only (Control) or CsA (2┌ g/mL) plus Y27632 (10┌ M) (Y72632) for 6 days. (FIG. 22B) Representative FACS analysis of cTnT+ CMs incubated with or without Y27632 (10┌ M). (FIG. 22C) % of cTnT+CM population incubated with or without Y27632 (10┌ M). Each group, n=4. ^{#}p<0.05 versus Control.

FIGS. 23A-23E. Combination of ROCK suppression and CsA additively promotes the differentiation and expansion of CMs from Flk1+ MPCs. (FIG. 23A) Self-beating area (inside area of white dotted line) in phase contrast images incubated with or without Y27632 (10┌ M). Scale bars represent 100┌ m. (FIG. 23B) Live cell images showing ┌ MHC-GFP+CMs in differentiating Flk1+ MPCs incubated with or without Y27632 (10┌ M). Scale bars represent 50┌ m. (FIG. 23C) Immunofluorescence images showing cTnT+CM and DAPI+ nuclei in differentiating Flk1+ MPCs incubated with or without Y27632 (10┌ M). Scale bars represent 50┌ m. (FIG. 23D) % of ┌ MHC-GFP+ area in live cell image incubated with or without Y27632 (10┌ M). n=3. *p<0.01 versus Control. (FIG. 23E) % of cTnT+ area in immunofluorescence image with or without Y27632 (10┌ M). n=4. *p<0.01 versus Control.

FIGS. 24A-24D. ROCK suppression increases number of αMHC-GFP+CMs in feeder-free condition. (FIG. 24A) Diagram of cell preparations for analyses of sorted ┌ MHC-GFP+CMs. ┌ MHC-GFP+CMs were further incubated with or without Y27632 (10┌ M) for 2 and 4 days, and analyses were performed at day 12.5 and 14.5. (FIG. 24B) Representative phase contrast images showing beating CMs incubated with or without Y27632 (10┌ M) at day 12.5. Scale bars represent 50┌ m. (FIG. 24C) Self-beating area (brown colored area) in phase contrast images incubated with or without Y27632 (10┌ M). Scale bars represent 50┌ m. (FIG. 24D) % of beating area incubated with or without Y27632 (10┌ M).

### DETAILED DESCRIPTION OF THE PREFERRED EMBODIMENTS

In the present application, “a” and “an” are used to refer to both single and a plurality of objects.

The meaning of “Rho-associated protein kinase (ROCK) suppressor” is well known in the art. An assay for determining a ROCK suppressor can include for example the following method. ROCKs were discovered as the first effectors of Rho and they induce the formation of stress fibers and focal adhesions by phosphorylating MLC (myosin light chain). Therefore, an assay for determining ROCK suppression is confirming MLC phosphorylation inhibition such as by Western blotting. In this regard, a ROCK suppressor, Y27632 inhibits MLC phosphorylation in Flk1+ MPCs during differentiation.

As used herein, “pluripotent cell” refers to refers to stem cells that can differentiate to all three germlines, endoderm, ectoderm and mesoderm, to differentiate into any cell type in the body, but cannot give rise to a complete organism. A totipotent stem cell is one that can differentiate or mature into a complete organism such as a human being.

Efficient differentiation and expansion of endothelial cells (ECs) and cardiomyocytes (CMs) from embryonic stem cell (ESC) is required for treating patients with cardiovascular diseases such as ischemia and severe congestive heart failure. Here, we fortuitously found that Rho-associated protein kinase (ROCK) inhibitor Y27632 profoundly promoted the differentiation and expansion of ECs and CMs from Flk1+ mesodermal precursor cells (MPCs), while reducing the differentiation and expansion of mural cells. The ROCK suppression-induced expansion of ECs and CMs appears to have resulted from promotion of proliferation of ECs and CMs via activation of PI3-kinase-Akt signaling. The ECs obtained by the combination of ROCK suppression and VEGF-A supplementation faithfully expressed most pan-EC surface markers, and phenotypic analyses revealed that they were differentiated toward arterial EC. Further incubation of the ICAM2+ ECs with Y27632 and VEGF-A for 2 days promoted expansion of ECs by 6.5-fold compared to those incubated with only VEGF-A. Importantly, the ROCK suppression-induced ECs displayed neovasculogenic abilities in vitro and in vivo. In addition, we found that ROCK inhibitor Y27632 with Cyclosporine A promoted CM differentiation and expansion from mouse ESC-derived Flk1+ MPCs in an OP9 co-culture system. Moreover, Y27632 increased the number of CMs in feeder-free condition. Thus, addition of ROCK inhibitor Y27632 during CM differentiation provides an efficient method for obtaining CM in a vast amount. Thus, supplementation of ROCK inhibitor Y27632 along with VEGF-A or Cyclosporine A provides a simple, efficient, and versatile method for obtaining ample amount of ESC-derived ECs or CMs at high purity suitable for use in therapeutic neovascularization for the patients with ischemia or in therapeutic cardiac cell implantation for the patients with congestive heart failure.

Neovascularization is a fundamental process that is essential not only for the development and maintenance of every organ, but also for the regeneration of failing organs. ECs derived from bone marrow, umbilical cord blood, and adipose tissue have been studied and applied for therapeutic neovascularization [48], and we have previously shown that freshly isolated donor stromal vascular fraction from adipose tissue can readily provide ECs that create vascular networks through “disassembly and reassembly” process, which can establish functional communication with the recipient circulation [45]. However, obtaining a sufficient amount of ECs from adult organ is hindered due to the low expansion capacity of the ECs.

Our 2D Matrigel system is a simple, versatile method that does not require feeder cells for differentiating Flk1+ MPC into several lineage precursor cells. Taking these advantages, we were able to dissect the downstream signaling pathways that underlie the VEGF-A-induced differentiation of ECs. In agreement with previous findings [8,11], we have confirmed PKA and PI3-kinase as the major downstream signaling pathways in the VEGF-A-induced differentiation and expansion of ECs. Meanwhile, we also observed that suppression of ROCK via Y27632 promotes the VEGF-A-induced differentiation and expansion of ECs by ˜4.2-fold, while the same profoundly suppressed the differentiation and expansion of ┌ SMA+MCs. Given that the Y27632-induced differentiation and expansion of ECs could be recapitulated through other ROCK inhibitors and siROCK in various ESC cell lines and iPSC, this ROCK suppression approach has a wide range of applicability. In addition, the simplicity of supplementing a small chemical compound Y27632 adds yet [49] another advantage to this protocol for procuring ample amount of ECs from ESC or iPSC for therapeutic neovascularization.

We further investigated how ROCK suppression via Y27632 is able to promote the differentiation and expansion of ECs from Flk1+ MPC. Our analyses revealed that the expansion of differentiated ECs by ROCK suppression was mainly derived from the combinative promotion of proliferation and adhesion of CD144+ ECs, rather than the suppression of apoptosis and anoikis of CD144+ ECs. In various types of adult ECs including HUVECs, activation of PI3-kinase/Akt signaling is mainly involved in cellular survival [49], while activation of ERK is mainly involved in cellular proliferation [50]. However, Y27632 did not activate ERK in the ECs in any condition, suggesting that the expansion of ECs did not result from ERK activation. Given that Y27632 activated Akt in the ECs—presumably through inactivation of PTEN [43]—and that blockade of PI3-kinase prevented the Y27632-induced differentiation and expansion of ECs, PI3-kinase/Akt signaling seems to be the major pathway in the proliferation of ECs derived from Flk1+ MPCs. Collectively, simultaneous VEGF-A- and ROCK suppression-induced Akt activation seems to contribute to the promotion of differentiation and expansion of ECs.

During embryonic development, ECs are further differentiated and specified into arterial, venous, and lymphatic ECs by various molecular regulations, and each specified EC displays diversity and heterogeneity at the molecular level, which are represented by respective surface markers [5,6,51,52]. Our phenotype analyses revealed that the ECs differentiated and expanded through VEGF-A stimulation and ROCK suppression faithfully expressed most pan-EC surface markers—CD31, CD144, Flk1, Tie2, endoglin, and ICAM2. Moreover, considering the fact that dll4, notch1, and claudin-5 were highly upregulated in these cells, it can be said that these ECs were further differentiated toward arterial ECs [5,51,52].

To maximize the number of ECs obtained from ESC, we expanded the ICAM2+ ECs for 2 days using the same system supplemented with VEGF-A and Y27632. In fact, not only did Y27632 promote adhesion ICAM2+ ECs onto the plate, but it also further expanded the ECs with VEGF-A through simultaneous promotion of cell proliferation and suppression of cell apoptosis. These ECs, which we referred to as VYI-ECs, showed that they had neovasculogenic potential in both in vitro and in vivo analyses. Furthermore, ROCK suppression positively promoted the neovasculogenic activities of VYI-ECs under VEGF-A stimulation, which is consistent with previous findings demonstrated in other EC types [53], but not with some reports [54-56], which could be due to differences in experimental designs or cell types. Our results show that VYI-ECs do have in vivo neovasculogenic capabilities.

Efficient Differentiation and Expansion of Endothelial Cells (ECs) and Cardiomyocytes

Large amount of CMs are lost after myocardial injuries such as acute myocardial infarction, and these injuries ultimately leads to congestive heart failure. Obtaining ample amount of purified CMs is an important goal of stem cell therapy aiming to implant differentiated CMs for cardiac regeneration. PSCs treated with cardiogenic small molecules provide good resources for cardiac regeneration. To date, various cardiogenic small molecules are found, but most of them are related with cardiogenic signaling pathways including BMP, TGF-beta/activin/NODAL, WNT and FGF [34]. Our discovery is that combination of ROCK suppression and CsA treatment additively promoted the differentiation and expansion of CMs from Flk1+ MPCs. The effect of CsA for CMs differentiation from PSCs was already demonstrated [36,37], but the effect of ROCK suppression for CMs differentiation has not been reported. We found that ROCK suppression under CsA treatment not only promoted differentiation of CMs from Flk1+ MPCs in an OP9 co-cultured condition, but also increased the number of CMs after sorting in feeder-free condition. These results implicated that RhoA-ROCK signaling is directly involved in CM differentiation and expansion.

Thus, supplementation of ROCK inhibitor Y27632 along with VEGF-A or Cyclosporine A provides a simple, efficient, and versatile method for obtaining ample amount of ESC-derived ECs or CMs.

There are many uses for techniques to stimulate the growth of endothelial cells and cardiomyocytes. Methods to manipulate the growth and/or differentiation of endothelial cells and cardiomyocytes would find uses in stem cell transplantation and regenerative medicine for patients. Growth of these cells may be carried out in vitro or ex vivo. For example, a patient's own cells could be expanded then re-introduced to the patient. Alternatively, ROCK suppressor agents may be introduced in vivo, either alone or in combination with other compatible agents or cells, e.g. at a site of tissue injury.

Administration and Dosage

When used therapeutically, the agents of the invention such as proteins, chemicals or cells are administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication.

The agent of the invention should be administered for a length of time sufficient to provide either or both therapeutic and prophylactic benefit to the subject. Generally, the agent is administered for at least one day. In some instances, the agent may be administered for the remainder of the subject's life. The rate at which the agent is administered may vary depending upon the needs of the subject and the mode of administration. For example, it may be necessary in some instances to administer higher and more frequent doses of the agent to a subject for example during or immediately following a event associated with tumor or cancer, provided still that such doses achieve the medically desirable result. On the other hand, it may be desirable to administer lower doses in order to maintain the medically desirable result once it is achieved. In still other embodiments, the same dose of agent may be administered throughout the treatment period which as described herein may extend throughout the lifetime of the subject. The frequency of administration may vary depending upon the characteristics of the subject. The agent may be administered daily, every 2 days, every 3 days, every 4 days, every 5 days, every week, every 10 days, every 2 weeks, every month, or more, or any time there between as if such time was explicitly recited herein.

Preferably, such agents are used in a dose, formulation and administration schedule which favor the activity of the agent and do not impact significantly, if at all, on normal cellular functions.

In one embodiment, the degree of activity of the agent is at least 10%. In other embodiments, the degree of activity of the drug is as least 20%, at least 30%, at least 40%, at least 50%, at least 60%, at least 70%, at least 80%, at least 90%, or at least 95%.

When administered to subjects for therapeutic purposes, the formulations of the invention are applied in pharmaceutically acceptable amounts and in pharmaceutically acceptable compositions. Such a pharmaceutical composition may include the agents of the invention in combination with any standard physiologically and/or pharmaceutically acceptable carriers which are known in the art. The compositions should be sterile and contain a therapeutically effective amount of the agent in a unit of weight or volume suitable for administration to a patient. The term “pharmaceutically acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V)

Suitable preservatives include benzalkonium chloride (0.003-0.03% W/V); chlorobutanol (0.3-0.9% W/V); parabens (0.01-0.25% W/V) and thimerosal (0.004-0.02% W/V)

A variety of administration routes are available. The particular mode selected will depend, of course, upon the particular combination of drugs selected, the severity of the disease condition being treated, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, other mucosal forms, direct injection, transdermal, sublingual or other routes. “Parenteral” routes include subcutaneous, intravenous, intramuscular, or infusion. Direct injection may be preferred for local delivery to the site of the cancer. Oral administration may be preferred for prophylactic treatment e.g., in a subject at risk of developing a cancer, because of the convenience to the patient as well as the dosing schedule.

Both non-biodegradable and biodegradable polymeric matrices can be used to deliver agents of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Thus, the invention provides a composition of the above-described agents for use as a medicament, methods for preparing the medicament and methods for the sustained release of the medicament in vivo.

The compositions may conveniently be presented in unit dosage form and may be prepared by any of the methods well known in the art of pharmacy. All methods include the step of bringing the therapeutic agents into association with a carrier which constitutes one or more accessory ingredients. In general, the compositions are prepared by uniformly and intimately bringing the therapeutic agent into association with a liquid carrier, a finely divided solid carrier, or both, and then, if necessary, shaping the product

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Other delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; liposomes; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation

Use of a long-term sustained release implant may be particularly suitable for treatment of established disease conditions as well as subjects at risk of developing the disease. “Long-term” release, as used herein, means that the implant is constructed and arranged to deliver therapeutic levels of the active ingredient for at least 7 days, and preferably 30-60 days. The implant may be positioned at a site of injury or the location in which tissue or cellular regeneration is desired. Long-term sustained release implants are well known to those of ordinary skill in the art and include some of the release systems described above

The therapeutic agent may be administered in alone or in combination with other agents including proteins, receptors, co-receptors and/or genetic material designed to introduce into, upregulate or down regulate these genes in the area or in the cells. If the therapeutic agent is administered in combination the other agents may be administered by the same method, e.g. intravenous, oral, etc. or may be administered separately by different modes, e.g. therapeutic agent administered orally, administered intravenously, etc. In one embodiment of the invention, the therapeutic agent and other agents are co-administered intravenously. In another embodiment the therapeutic agent and other agents are administered separately.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

### Example 1.1

### Cell Culture and Reagents

E14tg2a ESC cell line was a generous gift from Dr. Jun K. Yamashita (Kyoto University), and R1 ESC cell line was purchased from American Type Cell Culture. Mouse iPSC derived from FVB strain was a generous gift from Drs. Hyun-Jai Cho and Hyo-Soo Kim (Seoul National University Hospital) [38]. To obtain Flk1+ MPCs, ESCs and iPSCs were cultured on 0.1% gelatin-coated plates at a density of 1-2×10³ cells/cm² in differentiation medium [┌−minimal essential medium (Invitrogen) supplemented with 10% fetal bovine serum (FBS)] without leukemia inhibitory factor (LIF). At day 4.5, the Flk1+ MPCs were purified by AutoMACS™Pro Separator (Miltenyi Biotec) with biotin-conjugated anti-mouse Flk1 antibody (clone AVAS12a1, eBioscience) and streptavidin MicroBeads (Miltenyi Biotec). The Flk1+MPCs were then subsequently plated onto Matrigel (BD Biosciences)-coated plates at a density of 1˜2×10⁴ cells/cm², and were cultured in differentiation medium supplemented with VEGF-A₁₆₅ (50 ng/ml, Peprotech). To purify the ECs from the differentiating Flk1+ cells, the cells were first dissociated and resuspended in HBSS/2% FBS, and were serially incubated with anti-mouse ICAM2 antibody (rat monoclonal, clone 3C4, Biolegend) for 10 min and with anti-rat IgG Microbead (Miltenyi Biotec) for 15 min. We then sorted the ICAM2+ cells by AutoMACS′Pro Separator (Miltenyi Biotec), and subsequent FACS analysis showed that ˜90% of the ICAM2+ cells were CD31+/CD144+ ECs. VEGF-Trap was prepared as previously described [39]. Various signaling inhibitors and activators including SB203580, PD98059, KT5720, Go6976, KT5823, LY294002, rapamycin, Y27632, SB431542, Rho-kinase inhibitor (RKI; H-1152, isoquinolinesulfonamide), and Rho-kinase inhibitor II (RKI-II; pyridyl urea) were purchased from Calbiochem and Sigma-Aldrich.

SB203580 has the following chemical structure:

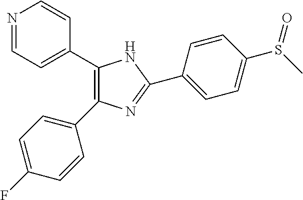 [SMILES: CS(=O)c1ccc(-c2nc(-c3ccc(F)cc3)c(-c3ccncc3)[nH]2)cc1]

PD98059 has the following chemical structure:

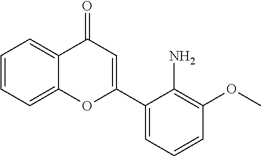 [SMILES: COc1cccc(-c2cc(=O)c3ccccc3o2)c1N]

KT5720 has the following chemical structure:

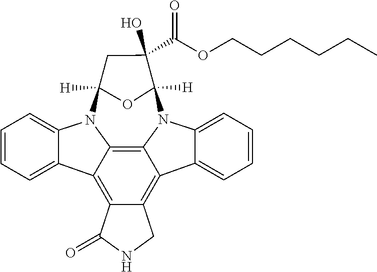 [SMILES: CCCCCCOC(=O)[C@@]1(O)C[C@H]2O[C@@H]1n1c3ccccc3c3c4c(c5c6ccccc6n2c5c31)C(=O)NC4]

Go6976 has the following chemical structure:

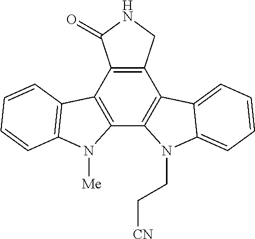 [SMILES: Cn1c2ccccc2c2c3c(c4c5ccccc5n(CCC#N)c4c21)CNC3=O]

KT5823 has the following chemical structure:

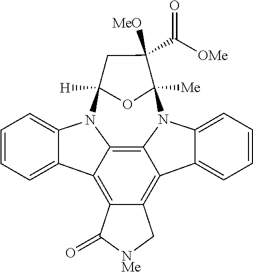 [SMILES: COC(=O)[C@@]1(OC)C[C@H]2O[C@]1(C)n1c3ccccc3c3c4c(c5c6ccccc6n2c5c31)C(=O)N(C)C4]

LY294002 has the following chemical structure:

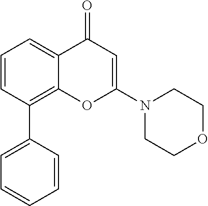 [SMILES: O=c1cc(N2CCOCC2)oc2c(-c3ccccc3)cccc12]

Y27632 has the following chemical structure:

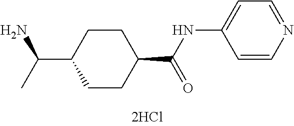 [SMILES: C[C@@H](N)[C@H]1CC[C@H](C(=O)Nc2ccncc2)CC1]

SB431542 has the following chemical structure:

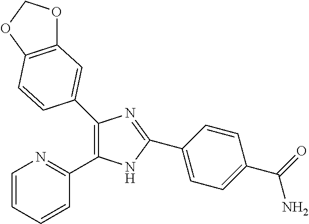 [SMILES: NC(=O)c1ccc(-c2nc(-c3ccc4c(c3)OCO4)c(-c3ccccn3)[nH]2)cc1]

EMG7 mouse ESCs which have ┌-myosin heavy chain (MHC) promoter driven enhanced green fluorescent protein (GFP) gene and OP9 cells were a generous gift from Dr. Jun K. Yamashita (Kyoto University). EMG7 ESCs were plated on a 0.1% gelatin coated 60p dish and maintained in a medium (GMEM, Invitrogen) containing 10% knockout serum, 2-mercaptoethanol (Invitrogen), sodium pyruvate (Invitrogen), non-essential amino acid and antibiotics (Invitrogen). leukemia inhibitory factor (LIF), Blasticidin (Invitrogen) and G418 (Invitrogen) were added in the medium at the time of the medium change. OP9 cells were maintained in a medium (alpha MEM, Invitrogen) containing 20% of fetal bovine serum (FBS), 2-mercaptoethanol (Invitrogen), L-glutamine (Invitrogen) and antibiotics (Invitrogen). The medium was changed every 2 days for both EMG7 ESCs and OP9 cells. For induction of Flk1+MPCs, EMG7 ESCs were cultured and plated on a 0.1% gelatin coated 100 p dish in the differentiation medium (alpha MEM, Invitrogen) with contained 10% FBS, 2-mercaptoethanol (Invitrogen), L-glutamine (Invitrogen) and antibiotics (Invitrogen) for 4.5 days (96-112 hours). The medium was changed every 2 days. At 4.5 days, differentiated EMG7 ESCs were harvested with 0.025% trypsin-EDTA and antigen recovery was performed in the differentiation medium for 30 minutes in an incubator. Then, cells were washed using phosphate buffered saline/10% FBS and attached with biotin-conjugated anti-mouse Flk1+ antibody (eBioscience) and anti-streptavidin MicroBeads (Miltenyi Biotec). Flk1+ MPCs were sorted by AutoMACS Pro Separator (Miltenyi Biotec). Sorted Flk1+ MPCs were plated onto the mitomycin-C (MMC) (Ag scientific) treated confluent OP9 cells at a density of 1-2×10⁴ cells/cm² and cultured in the differentiation medium. The medium was changed every 2 days. CsA (a gift from Novartis Pharma, Korea) was treated in sorted Flk1+ MPCs on OP9 cells. CsA was dissolved in dimethyl sulfoxide (DMSO) (Sigma) and diluted 2┌ g/mL in the differentiation medium at the time of medium change. 10┌ M of Y27632 (purchased from Calbiochem) were treated in sorted Flk1+MPCs on OP9 cells and sorted MHC GFP+CMs at the time of medium change.

### Example 1.2

### Immunofluorescence Staining of Cultured Cells

Cultured cells were fixed with 2% paraformaldehyde (PFA) and blocked with 5% goat (or donkey) serum in PBST (0.3% Triton X-100 in PBS) for 1 hr at room temperature (RT). The cells were incubated overnight at 4┌ C with the following primary antibodies: anti-mouse CD144 (clone 11D4.1, BD Pharmingen™), anti-mouse phospho-histone H3 (rabbit polyclonal, Upstate), anti-mouse cTnT (NeoMarkers), Cy3-conjugated anti-┌ SMA (clone 1A4, Sigma), and anti-mouse CD31 (clone 2H8, Chemicon). After washing in PBST 4 times, the cells were incubated for 2 hrs at RT with the following secondary antibodies: Cy3-conjugated anti-rabbit IgG (Jackson ImmunoResearch), Cy3-conjugated anti-hamster IgG (Jackson ImmunoResearch), Cy3-conjugated anti-mouse IgG antibody (Jackson ImmunoResearch) and FITC or Cy3-conjugated anti-rat (Jackson ImmunoResearch). F-actin and nuclei were stained with TRITC-phalloidin (Invitrogen) and 4′,6-diamidino-2-phenylindole (DAPI, Invitrogen), respectively. The cells were then mounted in fluorescent mounting medium (DAKO Corporation).

Immunofluorescent images were acquired using Zeiss LSM510 confocal fluorescence microscope (Carl Zeiss). Live images of ┌ MHC GFP+CMs were obtained using Axiovert 200M microscope (Carl Zeiss) equipped with AxioCam MRm (Carl Zeiss). Phase-contrast images including beating areas of CMs and sorted single CMs were obtained using an Infinity X digital camera and DpxView LE software (DeltaPix).

### Example 1.3

### Flow Cytometry and Cell Sorting

Cells were harvested with 0.25% trypsin-EDTA (Invitrogen) or dissociation buffer (Invitrogen) and resuspended in HBSS/2% FBS at 1×10⁶ cells per 100┌ l. The cells were incubated for 20 min with the following antibodies: biotin-conjugated anti-mouse Flk1 (clone AVAS12a1, eBioscience), allophycocyanin (APC)-conjugated anti-mouse CD140a (clone APA5, eBioscience), phycoerythrin(PE)-conjugated anti-mouse CD31 (clone 390, eBioscience), Alexa Fluor® 647-conjugated anti-mouse CD144 (clone BV13, eBioscience), PE-conjugated anti-mouse D114 (clone HMD4-1, Biolegend), PE-conjugated anti-mouse Tie2 (clone TEK4, eBioscience), PE-conjugated anti-mouse Endoglin (clone MJ7/18, Biolegend), anti-mouse ICAM2 (clone 3C4, Biolegend), APC-conjugated anti-mouse CD140b (clone APBS, eBioscience), PE-conjugated anti-mouse LYVE1 (clone ALY7, eBioscience), anti-mouse VEGFR3 (goat polyclonal, R&D systems), anti-mouse EphB4 (clone VEB4-7E4, Hycult biotech), and PE-conjugated anti-mouse Notch1 (clone HMN1-12, Biolegend). After washing in HBSS/2% FBS twice, the cells were incubated with the following secondary antibodies: PE-conjugated streptavidin (eBioscience), PE-conjugated anti-rat IgG2a (eBioscience), and FITC-conjugated anti-goat IgG (Invitrogen). Dead cells were excluded by 7-aminoactinomycin D (7-AAD, Invitrogen).

Analysis and sorting for CMs were performed at day 10.5. We harvested differentiated Flk1+ MPCs on OP9 cells with 0.25% trypsin-EDTA or dissociation buffer (Invitrogen). Cardiac troponin-T (cTnT)+ and ┌ smooth muscle actin (SMA)+ cells were analyzed after permeabilization using Cytofix/Cytoperm solution (BD Biosciences) for 15 min. After permeabilization, the cells were incubated for 30 min with anti-mouse cTnT (NeoMarkers) monoclonal antibody. After washing in 10% Perm/Wash buffer (BD Biosciences), the cells were incubated for 10 min with Alexa Fluor® 647 anti-mouse IgG (Invitrogen). After washing in 10% Perm/Wash buffer (BD Biosciences), the cells were incubated for 30 min with Cy3-conjugated anti-┌ SMA (clone 1A4, Sigma). We sorted out CMs using ┌ MHC GFP. Sorted Flk1+ MPCs on OP9 cells were harvested with 0.25% trypsin-EDTA or dissociation buffer (Invitrogen) and cells were washed using phosphate buffered saline/10% FBS twice. Dead cells were excluded using 7-aminoactinomycin D (Invitrogen). Then, we sorted out ┌ MHC GFP+CMs and plated on to 0.1% gelatin coated dish in the differentiation medium. The medium was changed every 2 days. Analyses and sorting were performed by FACS Aria II (Beckton Dickinson). Data were analyzed using FlowJo Version 7.5.4 software (TreeStar).

### Example 1.4

### EC Colony Assay

The Flk1+ MPCs were plated onto Matrigel-coated plate at a density of 4,000 cells/cm², and cultured in differentiation medium supplemented with VEGF-A₁₆₅ (50 ng/ml) and Y27632 (10┌M). At 36 hrs later, the number of CD144+ EC colonies was counted. Clustering of >5 CD144+ ECs was regarded as one EC colony.

### Example 1.5

### Angiogenesis Assay Using a Microfluidic Device

To test the angiogenic property of VYI-ECs, we utilized a microfluidic platform that was modified from the previously described device [40,41]. Briefly, a poly-dimethylsiloxane (PDMS) based microfluidic platform containing three channels was fabricated with poly-dimethylsiloxane using a soft lithography process. Two empty spaces in between three parallel channels were filled with a mixture of type I collagen (3 mg/ml, rat tail, BD Biosciences) and fibronectin (50 μg/ml, Invitrogen), which together acted as an ECM scaffold. The center channel was coated with fibronectin (10 μg/ml), and was seeded with 40˜50 μl of VYI-EC suspension (1×10⁷ cells/ml). The device was tilted vertically for 1 hr to allow the cells to attach onto the sidewall of the scaffold by gravity. The cells were then seeded onto the other sidewall by flipping the device upside down. Cell culture media in the side channels containing VEGF-A (50 ng/ml) or VEGF-A (50 ng/ml) plus Y27632 (10 μM) were exchanged with fresh media every 12 hr. Phase contrast and immunofluorescent images were acquired to analyze the angiogenic sprouting formation of VYI-ECs.

### Example 1.6

### In Vivo Matrigel Plug Assay

1×10⁶ VYI-ECs procured from iPSC (FVB strain) [38] were mixed with 100┌ l Matrigel supplemented with VEGF-A (500 ng/ml), and the mixture was implanted subcutaneously into the dorsal side of six-weeks-old Tie2-GFP mice (FVB strain). Animal care and experimental procedure were performed with the approval of the Animal Care Committee of KAIST. After three weeks, the mice were sacrificed after injection of anesthetic mixture (80 mg/kg ketamine and 12 mg/kg xylazine), and the implanted Matrigel was fixed by systemic vascular perfusion with 1% PFA, harvested, and whole-mounted for histologic analyses.

### Example 1.7

### Assays for Apoptosis and Cell Cycle

Apoptosis assay was performed as previously described [12]. To determine the cell cycle, BrdU/7-AAD cell cycle analysis was performed according to the manufacturer's instructions (BD Pharmingen™). Briefly, cells were incubated with BrdU (1 mM) for 1 hr, dissociated with dissociation buffer, and stained with anti-CD144 antibody. The cells were then fixed, permeabilized, and fixed once more, followed by 1 hr incubation with DNase I (200 U) at 37┌ C. After incubation with FITC-conjugated anti-BrdU antibody for 20 min at RT, the cells were stained with 7-AAD. The cells were analyzed by FACS Aria II and the data were analyzed using FlowJo software.

### Example 1.8

### Cell Adhesion Assay

Sorted Flk1+ MPCs or ICAM2+ ECs were plated onto Matrigel coated plates at a density of 10,000 cells/cm². Six hrs after plating, non-adherent cells were removed by washing and the cell number of adherent cells was counted following trypsinization. The percentage of adherent cells was calculated as (counted cell number/initial seeded cell number)×100.

### Example 1.9

### Quantitative RT-PCR

Total RNA from purified ICAM-2+ ECs was extracted using Trizol RNA extraction kit (Invitrogen) according to the manufacturer's instructions. 1┌ g of the RNA was reverse transcribed into cDNA using GoScript™ cDNA synthesis system (Promega). Synthesized cDNA was applied for quantitative real-time PCR using FastStart SYBR green Master (Roche) and Bio-Rad S1000 Thermal cycler with the indicated primers (Table I). GAPDH was used as reference gene and the results were presented as relative expression to control using the ddCt method [42].

TABLE 1
Quantitative RT-PCR Primers

| | | |
|---|---|---|
| vegfr-1 | Forward | 5′- GGC TCT ACG ACC TTA GAO TGT CA -3′ (SEQ ID NO: 1) |
| | Reverse | 5′- TGC TGT TTC CTG GTC CTA AAA TA -3′ (SEQ ID NO: 2) |
| vegfr-2 | Forward | 5′- ACC AGA AGT AAA AGT GAT CCC AGA -3′ (SEQ ID NO: 3) |
| | Reverse | 5′- TCC ACC AAA AGA TGG AGA TAA TTT -3′ (SEQ ID NO: 4) |
| vegfr-3 | Forward | 5′- GGT GTC ACC TTC GAC TGG GAT TAT CC -3′ (SEQ ID NO: 5) |
| | Reverse | 5′- CAT TCT GGC TGA CAT TGT GGA TGG TC -3′ (SEQ ID NO: 6) |
| vegf-a | Forward | 5′- CAG AAG GAG AGC AGA AGT CC -3′ (SEQ ID NO: 7) |
| | Reverse | 5′- CTC CAG GGC TTC ATC GTT A -3′ (SEQ ID NO: 8) |
| vegf-c | Forward | 5′- CGT TCT CTG CCA GCA ACA TTA CCA C -3′ (SEQ ID NO: 9) |
| | Reverse | 5′- CTT GTT GGG TCC ACA GAC ATC ATG G -3′ (SEQ ID NO: 10) |
| vegf-d | Forward | 5′- GTC TGT AAA GCA CCA TGT CCG GGA G -3′ (SEQ ID NO: 11) |
| | Reverse | 5′- CCA CAG GCT GGC TTT CTA CTT GCA C -3′ (SEQ ID NO: 12) |
| tie-1 | Forward | 5′- AGA CTG ACG TGA TCT GGA AGA AC -3′ (SEQ ID NO: 13) |
| | Reverse | 5′- ACT TGA TGG TGG CTG TAC ATT TT -3′ (SEQ ID NO: 14) |
| tie-2 | Forward | 5′- GCC ATC AAG AGG ATG AAA GAG TAT -3′ (SEQ ID NO: 15) |
| | Reverse | 5′- GCT CCC AAG AGA TTA ATG ATG TTT -3′ (SEQ ID NO: 16) |
| nrp1 | Forward | 5′- AGG TTT CTC AGC CAA CTA CAG TG -3′ (SEQ ID NO: 17) |
| | Reverse | 5′- ACT GTG AAG ATG CAG TGA TCT GA -3′ (SEQ ID NO: 18) |
| nrp2 | Forward | 5′- GCA GGT TTC TCT CTA CGC TAT GA -3′ (SEQ ID NO: 19) |
| | Reverse | 5′- ATA CTT CTC TGG AAA CCC TGG AG -3′ (SEQ ID NO: 20) |
| ephb2 | Forward | 5′- AAG ATC CTC ATG AAA GTT GGA CA -3′ (SEQ ID NO: 21) |
| | Reverse | 5′- TGA ACT TCT CCC ATT TGT ACC AG -3′ (SEQ ID NO: 22) |
| ephb4 | Forward | 5′- AAG ATG GGA AGA TAC GAG GAA AG -3′ (SEQ ID NO: 23) |
| | Reverse | 5′- CAA GAT TTT CTT CTG GTG TCC TG -3′ (SEQ ID NO: 24) |
| dll4 | Forward | 5′- GAA TGT ATC CCC CAC AAT GGC TGT CG -3′ (SEQ ID NO: 25) |
| | Reverse | 5′- GCA CGG AGA GTG GTG AGT ACA GTA -3′ (SEQ ID NO: 26) |
| notch1 | Forward | 5′- CTG TGA GAC CAA CAT CAA TGA GTG C -3′ (SEQ ID NO: 27) |
| | Reverse | 5′- GCA GTC ATC CAG GTT GAT CTC ACA G -3′ (SEQ ID NO: 28) |
| cdh5 | Forward | 5′- CCA ACA TGC TAC CTG CCC ACC ATC -3′ (SEQ ID NO: 29) |
| | Reverse | 5′- GGG CAG TAA GGA AGT ACT CAG AGA C -3′ (SEQ ID NO: 30) |
| claudin5 | Forward | 5′- CTG GTG GCA CTC TTT GTT ACC TTG AC -3′ (SEQ ID NO: 31) |
| | Reverse | 5′- CCA GCT CGT ACT TCT GTG ACA CC -3′ (SEQ ID NO: 32) |
| etv2 | Forward | 5′- CAG AGT CCA GCA TTC ACC AC -3′ (SEQ ID NO: 33) |
| | Reverse | 5′- AGG AAT TGC CAC AGC TGA AT -3′ (SEQ ID NO: 34) |
| ets1 | Forward | 5′- GTG GAT CTC GAG CTT TTC CCT TCC -3′ (SEQ ID NO: 35) |
| | Reverse | 5′- CCG GGG GTC TTT GGG GAT TC -3′ (SEQ ID NO: 36) |
| ets2 | Forward | 5′- GCC TGG ATT CTG TCT CCC ATG ACT C -3′ (SEQ ID NO: 37) |
| | Reverse | 5′- CAG CCA GGG GTT TTT GGG GAT G -3′ (SEQ ID NO: 38) |
| sox7 | Forward | 5′- GGG GTC CAT CTC AGT TCA GTC TCT AAC -3′ (SEQ ID NO: 39) |
| | Reverse | 5′- CTT GAG AGG GCT GTA ACT TGG AGA TCC-3′ (SEQ ID NO: 40) |
| sox17 | Forward | 5′- GAG GGC CAG AAG CAG TGT TAC AC -3′ (SEQ ID NO: 41) |
| | Reverse | 5′- CTG GCT AAA ACT GGA CAG TGA TTG TG -3′ (SEQ ID NO: 42) |
| sox18 | Forward | 5′- GCA AGC GAC TGG CGC AAC AAA ATC -3′ (SEQ ID NO: 43) |
| | Reverse | 5′- CGG TAC TTG TAG TTG GGA TGG TCG C -3′ (SEQ ID NO: 44) |
| sm22a | Forward | 5′- TCC AGG TGT GGC TGA AGA ATG GTG -3′ (SEQ ID NO: 45) |
| | Reverse | 5′- CTG TGA AGT CCC TCT TAT GCT CCT G -3′ (SEQ ID NO: 46) |
| calponin | Forward | 5′- GCA TCG GGA ACA ACT TCA TGG ATG G -3′ (SEQ ID NO: 47) |
| | Reverse | 5′- CTC TGC ATA CTT GAC TCC CAC ATT GAC -3′ (SEQ ID NO: 48) |
| sma | Forward | 5′- CAT CGT GGG ACG TCC CAG ACA TCA -3′ (SEQ ID NO: 49) |
| | Reverse | 5′- ACA GCC TGA ATA GCC ACA TAC ATG GC -3′ (SEQ ID NO: 50) |
| gapdh | Forward | 5′- GTC GTG GAG TCT ACT GGT GTC TTC AC -3′ (SEQ ID NO: 51) |
| | Reverse | 5′- GTT GTC ATA TTT CTC GTG GTT CAC ACC C -3′ (SEQ ID NO: 52) |

### Example 1.10

### Western Blotting

Cell lysates were separated by SDS-PAGE gel and transferred to PVDF membranes for Western Blot analysis. After blocking with 5% skim milk, the membranes were first incubated with primary antibodies in blocking buffer overnight at 4┌ C, then with HRP-conjugated secondary antibodies for 1 hr at RT. The following primary antibodies were used: anti-RhoA (mouse monoclonal, Cytoskeleton Inc.), anti-ROCK1 (mouse monoclonal, Santa Cruz), anti-ROCK2 (rabbit polyclonal, Santa Cruz), anti-┌-tubulin (mouse monoclonal, Santa Cruz), anti-phospho-PTEN (Ser380/Thr382/383)(clone 44A7, Cell Signaling), anti-PTEN (clone 138G6, Cell Signaling), anti-phospho-Akt (Ser473)(clone 193H12, Cell Signaling), anti-Akt (rabbit polyclonal, Cell Signaling), anti-phospho-ERK (Thr202/Tyr204) (clone 20G11, Cell Signaling), anti-ERK (rabbit monoclonal, Cell Signaling), anti-phospho-MyPT (Thr696) (rabbit polyclonal, Cell Signaling), anti-MyPT (rabbit polyclonal, Cell Signaling), anti-phospho-MLC (Thr18/Ser19) (rabbit polyclonal, Cell Signaling), anti-MLC (rabbit polyclonal, Cell Signaling), anti-┌ SMA (rabbit polyclonal, Novus Biologicals), anti-GAPDH (rabbit polyclonal, Santa Cruz), anti-phospho-VEGFR2 (Tyr1175) (clone 19A10, Cell Signaling), and anti-VEGFR2 (clone 55B11, Cell Signaling). The secondary antibodies used were rabbit anti-mouse IgG-HRP (Santa Cruz) and goat anti-rabbit IgG-HRP (Santa Cruz). Signals were developed with enhanced chemiluminescence HRP substrate (Millipore) and detected using LAS-3000 mini (Fuji film).

### Example 1.11

### siRNA Knockdown of ROCK1 and ROCK2

Purified Flk1+ MPCs were plated on Matrigel-coated plates on day 4.5 and incubated with differentiation medium. After 6 hrs, the cells were transfected with siGENOME SMARTpool siRNA against ROCK1, ROCK2, or non-specific control siRNA (each 80 nM, Dharmacon) using RNAi Max transfection reagent (Invitrogen) according to the manufacturer's instruction. After 6 hrs of transfection, the cells were incubated with differentiation medium supplemented with VEGF-A (50 ng/ml). Analysis of differentiation of ECs was performed 36 hrs after the transfection.

### Example 1.12

### Matrigel Tube Forming Assay

The purified VYI-ECs (described in detail in the Results section) were plated on Matrigel-filled plates at a density of 2-4×10⁴ cells/cm², and were incubated in differentiation medium containing VEGF-A (50 ng/ml) with or without Y27632 (10┌ M). After 12 hrs, the tube structures were observed using Axiovert 25 microscope (Carl Zeiss) and phase-contrast images were taken using DeltaPix infinity X digital camera and DpxView LE software (DeltaPix, Denmark).

### Example 1.13

### Scratch Wound Healing Assay

The purified VYI-ECs were plated on Matrigel-coated plates and incubated in differentiation medium until confluent. A single scratch wound was made on the cell-plated surface with a 200┌ l disposable pipette tip, and the medium was exchanged with a new medium containing either VEGF-A (50 ng/ml), Y27632 (10┌ M), or both. The wound was allowed to heal for 12 hrs. Phase contrast images were taken at 0, 3, 6, and 12 hrs. The average extent of wound closure was evaluated by measuring the wound area using ImageJ software.

### Example 1.14

### Statistics

Values presented are means ┌ standard deviation (SD). Significant differences between the means were determined by analysis of variance followed by the Student-Newman-Keuls test. Significance was set at p<0.05 or 0.01.

### Example 2

### Results

### Example 2.1

### ROCK Suppression Promotes the Differentiation and Expansion of ECs from Flk1+ MPCs

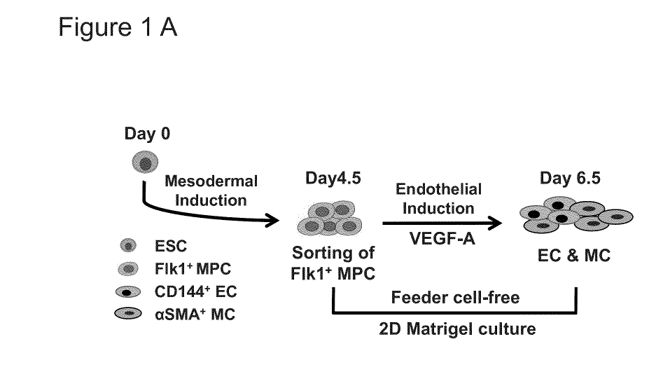

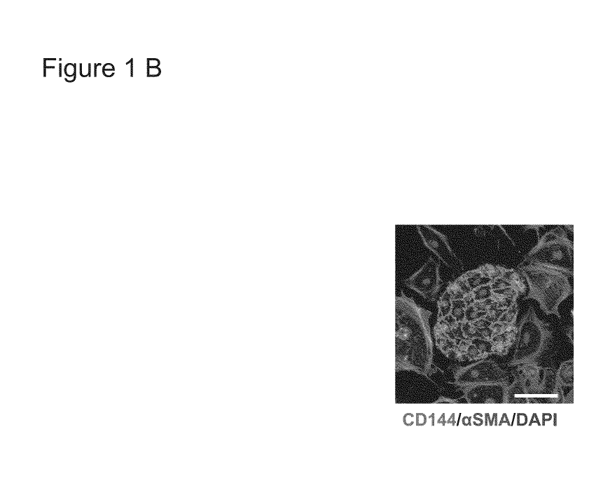

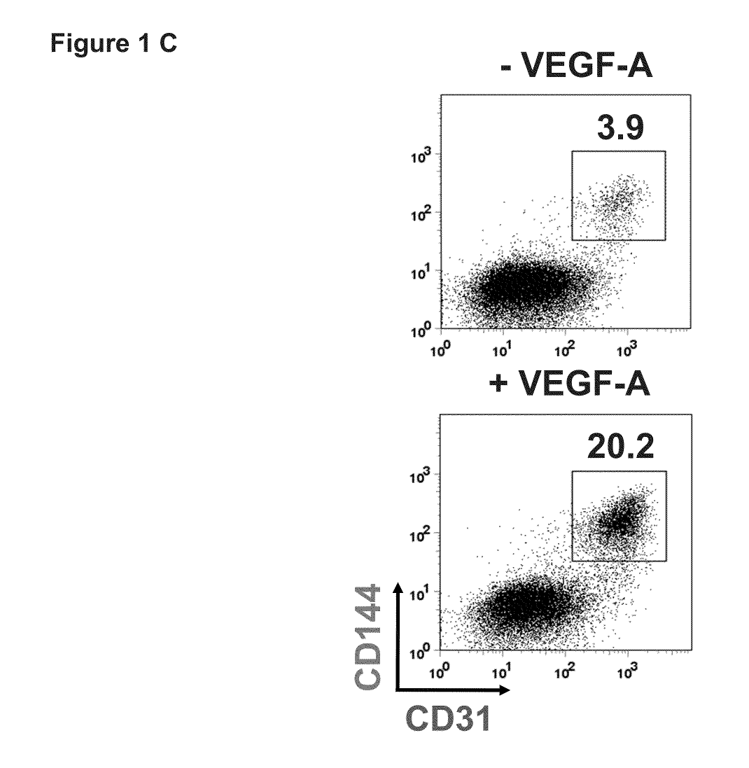

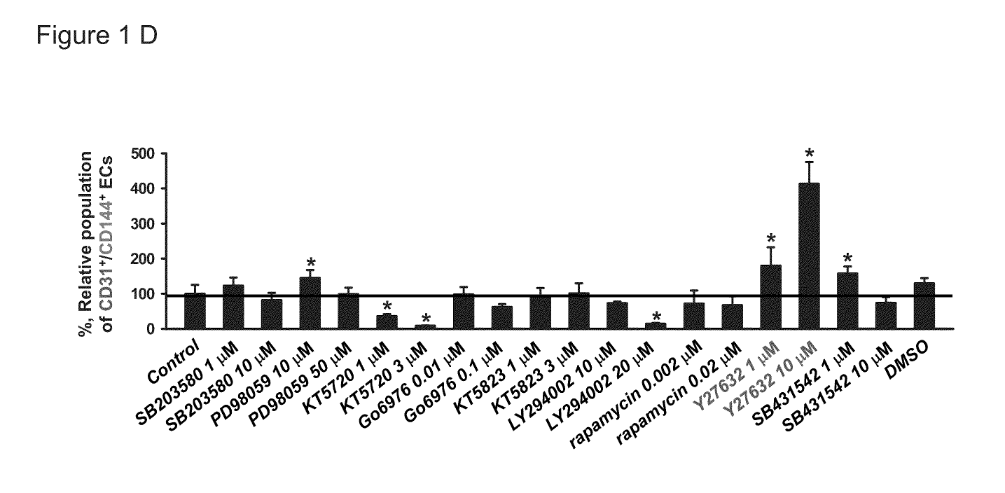

Flk1+ MPCs derived from mouse ESCs are capable of differentiating into ECs, hematopoietic cells, mural cells (MCs), and cardiomyocytes [1,4]. To avoid cell contamination from using feeder cells for differentiation of Flk1+ MPCs, we have established a feeder cell-free 2D Matrigel system (FIG. 1A). Flk1+ MPCs were differentiated from E14tg2a ESC cell line, and when the Flk1+ MPCs were purified at day 4.5 and cultured on Matrigel-coated plates in the differentiation medium, colonies of CD144+ ECs surrounded by ┌ SMA+MCs were formed (FIG. 1B). At day 6.5, population of CD144+/CD31+ ECs grown in differentiation medium was ˜4%, whereas the same EC population was increased up to ˜20% when VEGF-A (50 ng/ml) was supplemented (FIG. 1C). For following experiments, we chose to use 50 ng/ml of VEGF-A (hereafter referred to as VEGF-A) based on its maximal effect on differentiation and expansion of ECs from Flk1+ MPCs.

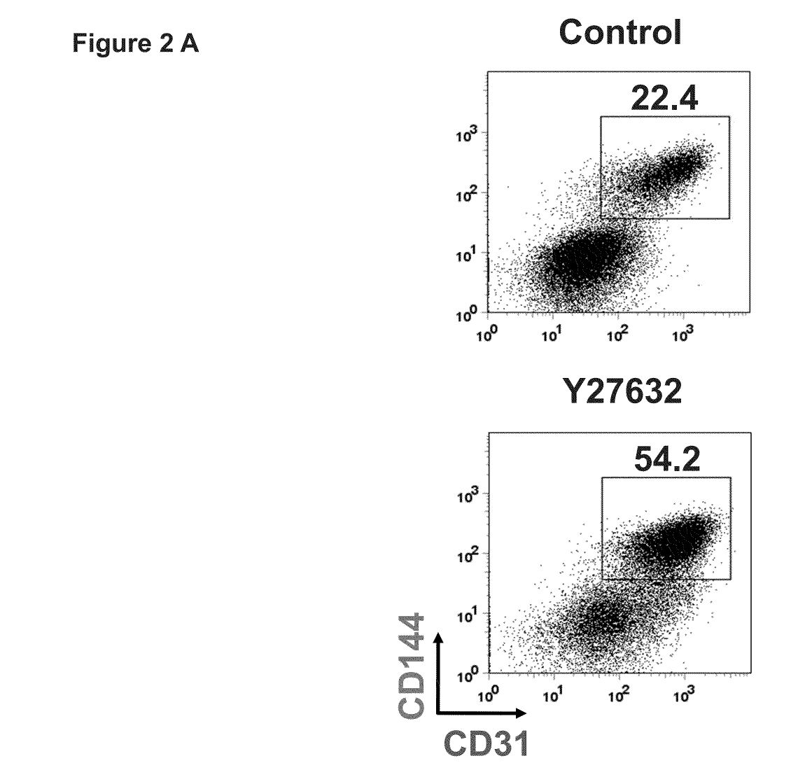

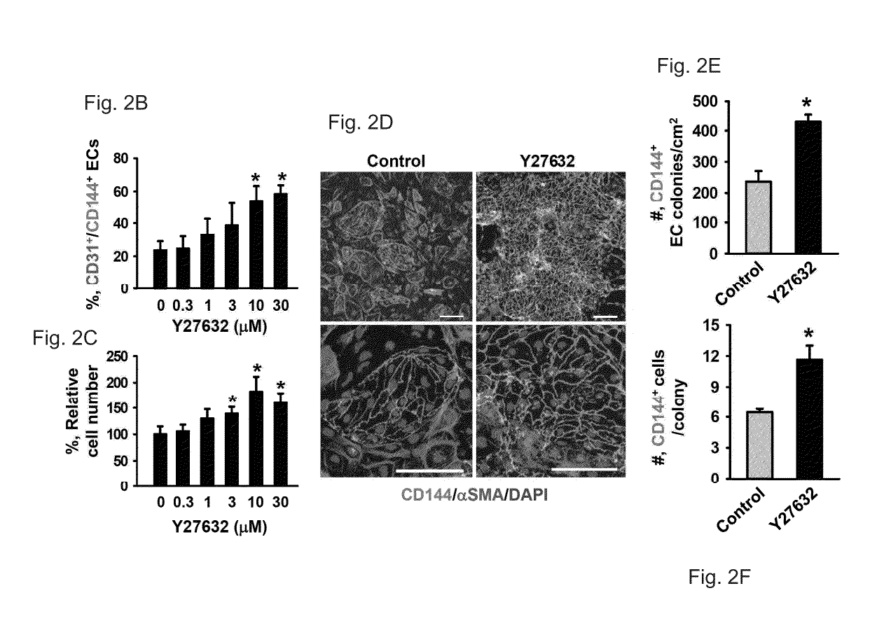

To dissect the signaling pathways involved in VEGF-A-induced differentiation and expansion of ECs, various kinds of signaling inhibitors—PI3-kinase inhibitor (LY294002), MEK/ERK inhibitor (PD98059), p38 inhibitor (SB203580), PKA inhibitor (KT5720), PKC inhibitor (Go6976), PKG inhibitor (KT5823), mTOR inhibitor (rapamycin), TGF-1 inhibitor (SB431542), and ROCK inhibitor (Y27632)—were treated with VEGF-A, and the population of CD31+/CD144+ ECs was analyzed at day 6.5 (FIG. 1D). Among them, PKA inhibitor and PI3-kinase inhibitor strongly reduced the CD31+/CD144+ EC population, indicating that the VEGF-A-induced differentiation of ECs mainly results from activation of PKA and PI3-kinase, which is in agreement with the previous report [8]. To our surprise, we noticed that ROCK inhibitor Y27632 (1 and 10┌ M) dramatically increased the CD31+/CD144+ EC population (1.8- and 4.1-folds respectively when compared to Control). Y27632 treatment simultaneously increased not only the percentage of EC population, but also the total cell number in a dose-dependent manner (FIGS. 2A-C). Accordingly, we were able to amplify the total number of CD31+/CD144+ ECs from Flk1+ MPCs by ˜4.2-fold through treatment of 10┌ M of Y27632 plus VEGF-A compared to treatment of only VEGF-A (Control). We chose 10┌ M of Y27632 (hereafter referred to as Y27632 otherwise indicated) for following experiments based on its maximum effect on the promotion of EC population. Immunofluorescent staining analysis on day 6.5 showed that Y27632 increased both the size and number of CD144+ EC colonies, while it decreased the number of surrounding ┌ SMA+MCs (FIG. 2D). In addition, EC colony assay revealed that Y27632 increased the number of EC colonies as well as the cell number in each colony (FIGS. 2E and 2F). These data indicate that Y27632 promotes not only the differentiation of ECs from Flk1+ MPCs but also the expansion of the induced ECs.

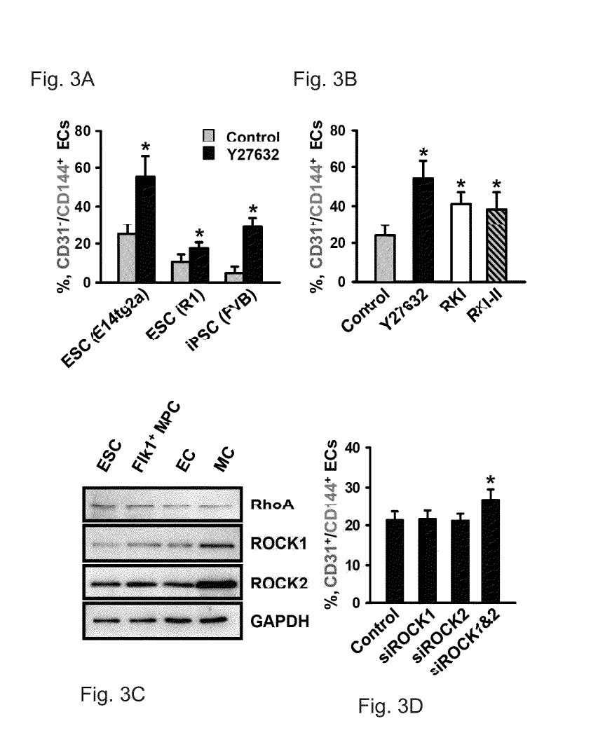

Aside from the E14tg2a cell line used above, Y27632 also promoted differentiation and expansion of ECs from R1 ESC cell line and iPSC by 1.6- and 5.9-fold compared to each control, respectively, suggesting that this procedure can be applied to Flk1+ MPCs derived from any source of mouse ESCs (FIG. 3A). To confirm that the effect of Y27632 is truly via suppression of ROCK signaling, we applied 2 different ROCK inhibitors—RKI and RKI-II; both RKI and RKI-II similarly increased EC population, indicating that the promotion of EC population is indeed a result of suppression of ROCK signaling (FIG. 3B). In fact, both ROCK1 and ROCK2 and their main upstream signaling molecule RhoA were expressed in ESCs and Flk1+ MPCs as well as in ECs and MCs (FIG. 3C). To find the ROCK subtype responsible for the increase in EC population, we depleted ROCK1, ROCK2, or both by treatment of their respective siRNAs into the Flk1+ MPCs. While treatment of either siROCK1 or siROCK2 alone did not promote EC population, the combination of siROCK1 and siROCK2 significantly promoted EC population (FIG. 3D). Our interpretation of this finding is that blockade of one subtype of ROCK up-regulates the other type of ROCK and negates the siRNA-mediated blocking effect, so that blockade of both types of ROCK is required for the promotion of EC population.

### Example 2.2

### Expansion of Differentiated ECs by ROCK Suppression is Mainly Due to the Promotion of EC Proliferation

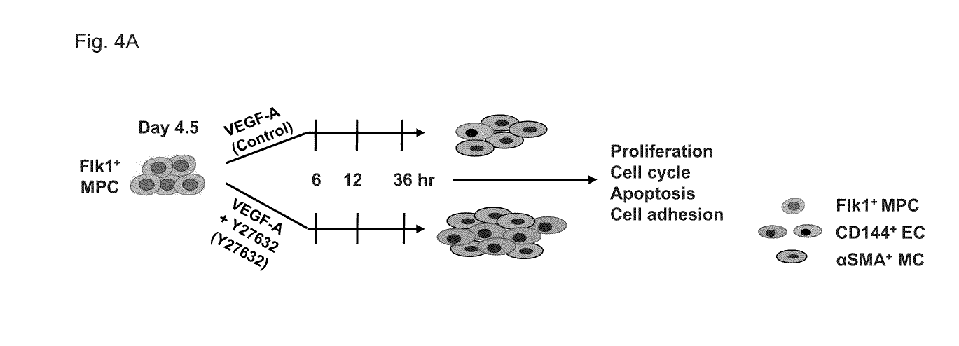

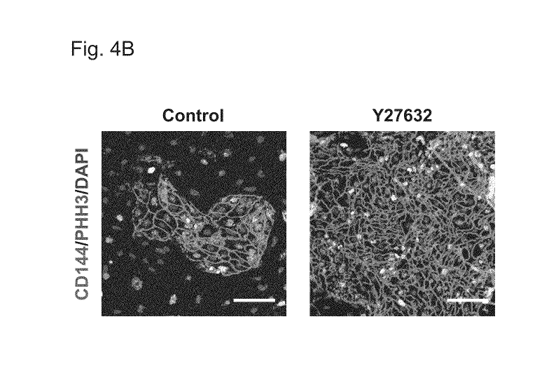

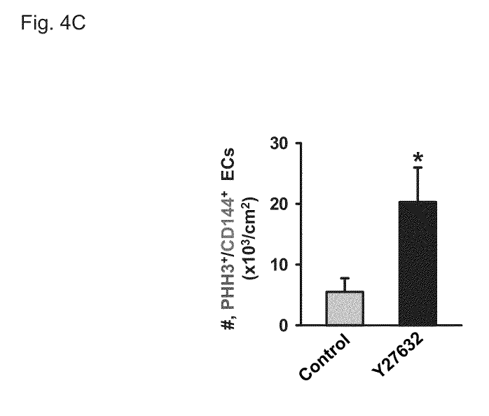

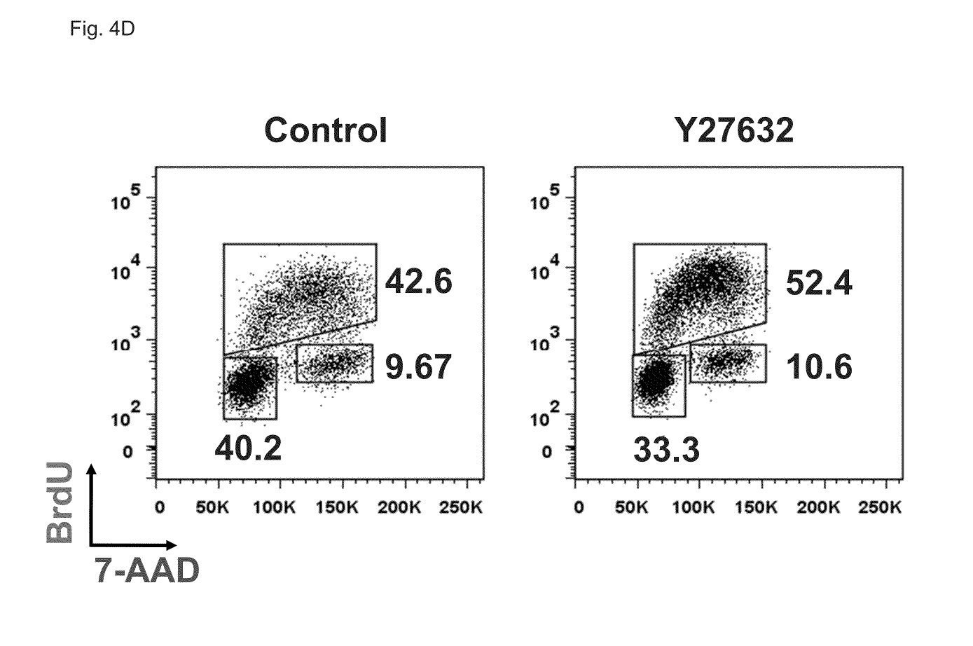

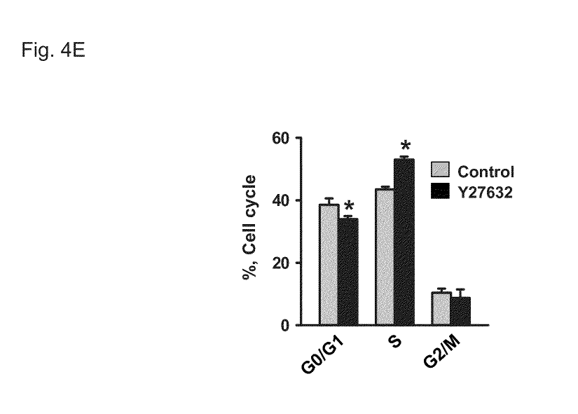

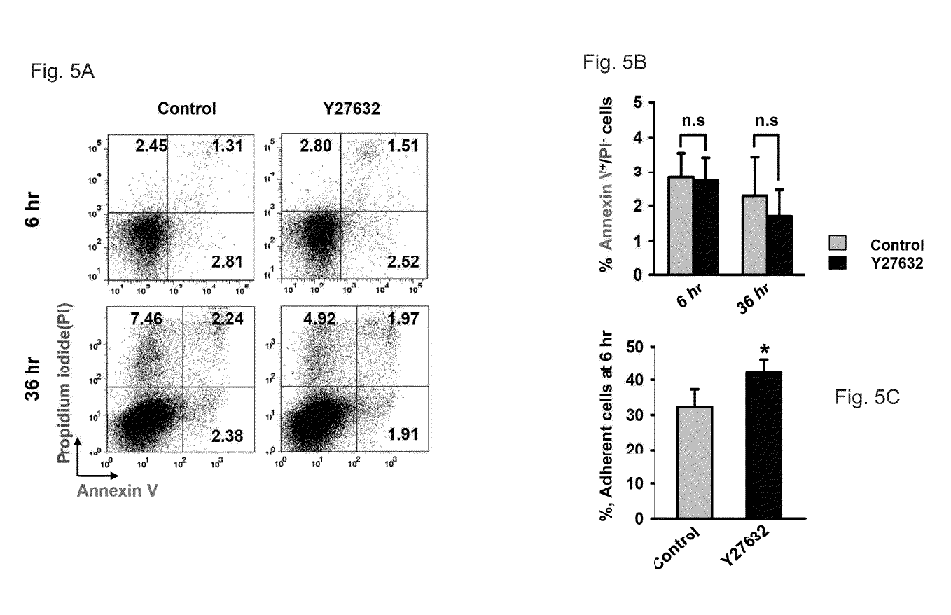

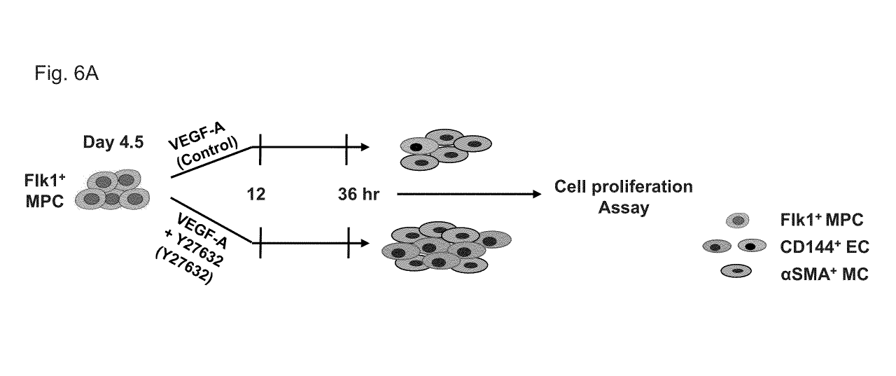

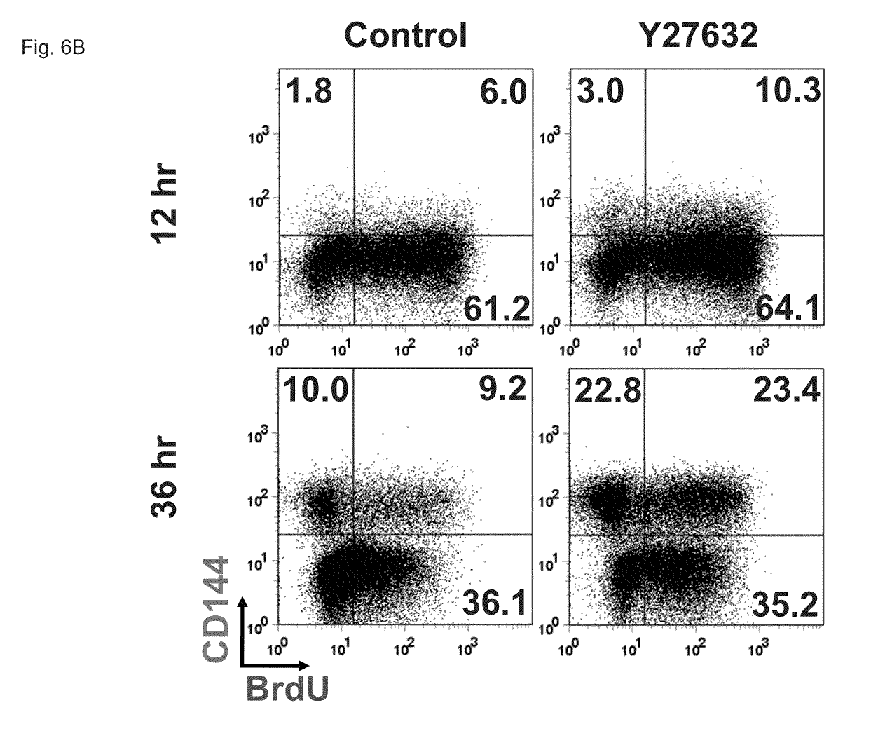

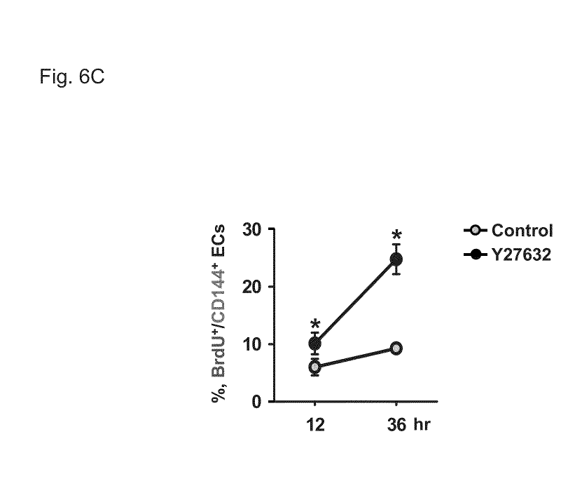

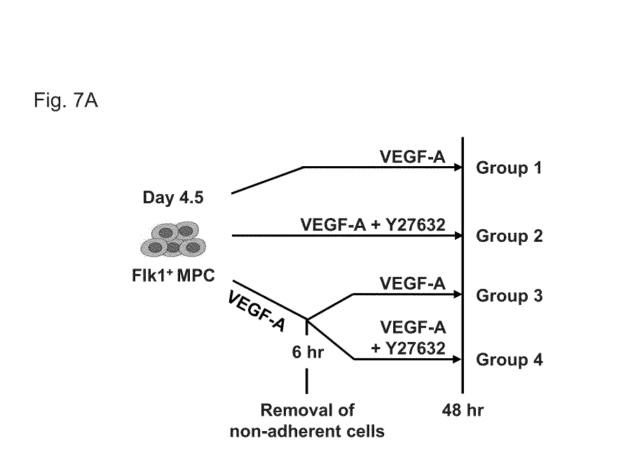

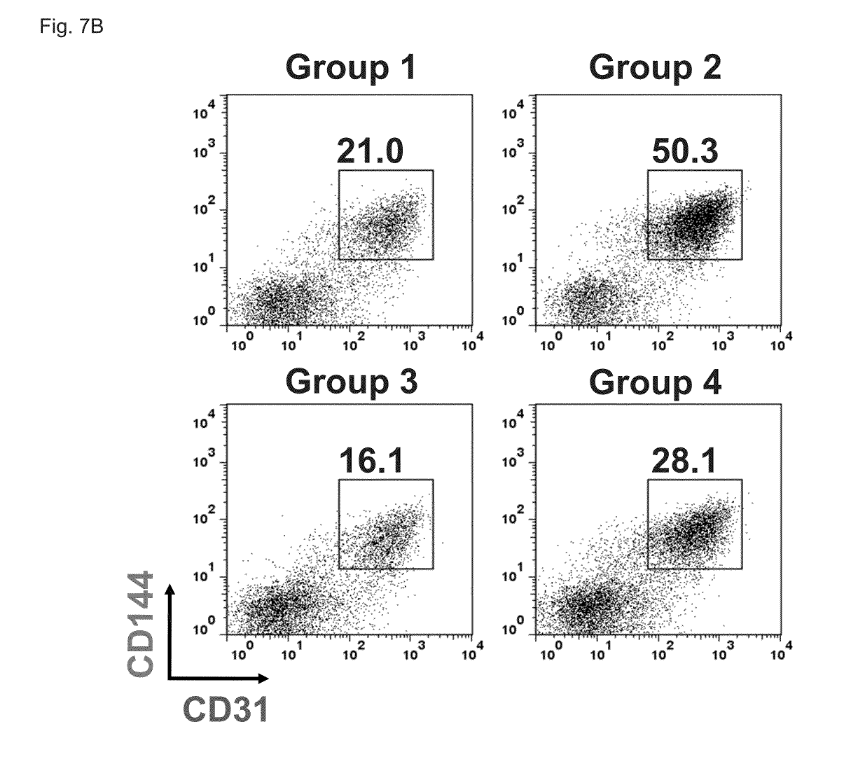

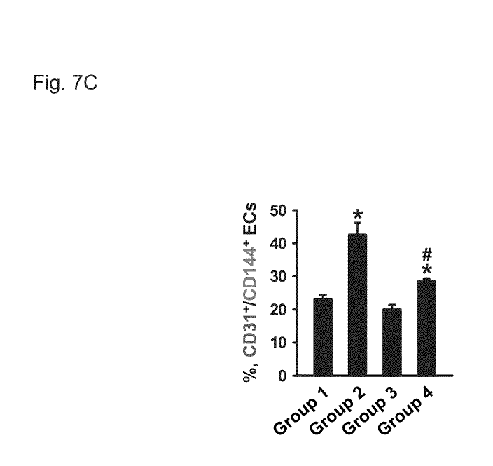

To elucidate how ROCK suppression promotes the differentiation and expansion of ECs, we examined the effect of Y27632 on the proliferation, dissociation-induced apoptosis (also regarded as “anoikis”) at 6 hrs, apoptosis at 36 hrs, and adhesion during differentiation (FIG. 4A). Phospho-histone H3 (PHH3) immunostaining also revealed that Y27632 increased the population of PHH3+/CD144+ ECs by 3.7-fold compared to control at 36 hrs (FIGS. 4B and 4C). Accordingly, BrdU/7-AAD analysis revealed that compared to control, Y27632 increased the population of CD144+ ECs in S phase by 1.23-fold, whereas it reduced those in G0/G1 phase by 12% at 36 hrs (FIGS. 4D and 4E). However, Y27632 did not significantly change the population of Annexin V+/propidium iodide (PI)-apoptotic cells at 6 and 36 hrs (FIGS. 5A and 5B). Notably, Y27632 promoted the adhesion of Flk1+ cells onto the plates by 1.3-fold compared to control at 6 hrs (FIG. 5C). BrdU incorporation assay revealed that during the differentiation process of ECs, Y27632 increased BrdU+/CD144+ EC population by 1.7-fold and 2.7-fold at 12 and 36 hrs, respectively (FIG. 6). We observed that, at 48 hrs after removal of the non-adherent cells at 6 hrs, Y27632 also simultaneously increased the percentage of EC population by 1.4-fold compared to control (FIG. 7). Thus, the expansion of differentiated EC by ROCK suppression is mainly due to the combinative promotion of proliferation and adhesion of CD144+ ECs, rather than the suppression of anoikis and apoptosis of the same.

### Example 2.3

### ROCK Suppression Promotes the Differentiation of ECs Through PTEN-Akt Signaling and Inhibits the Differentiation of MCs Through MyPT-MLC Signaling

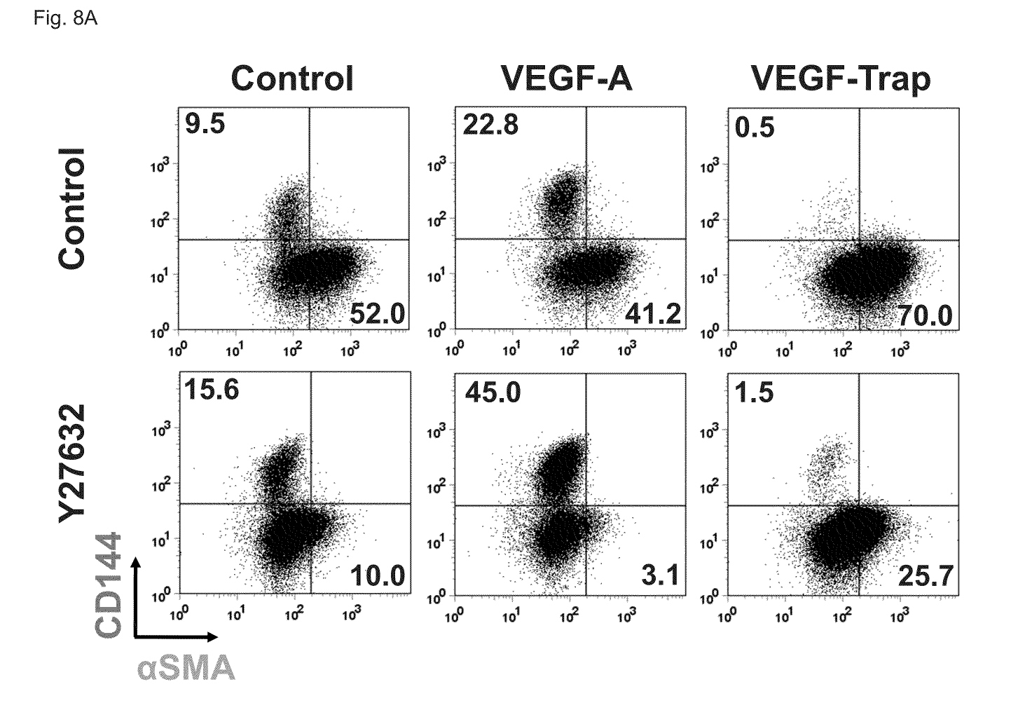

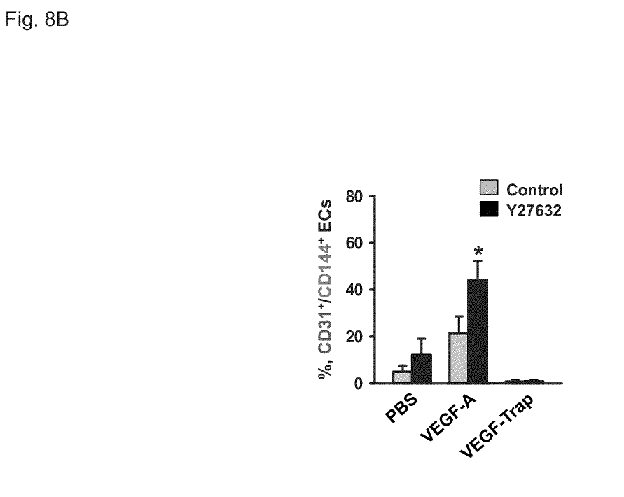

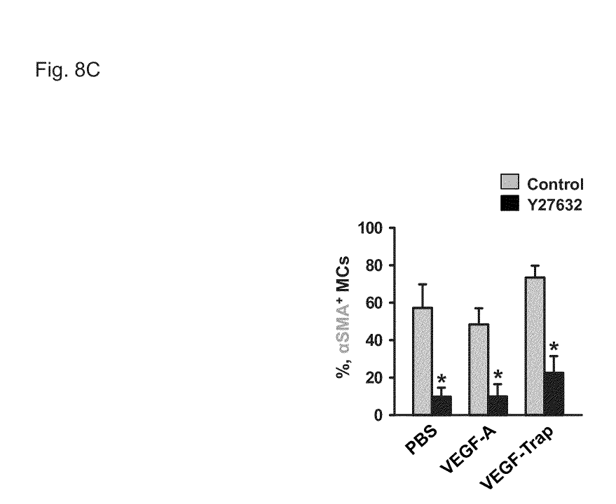

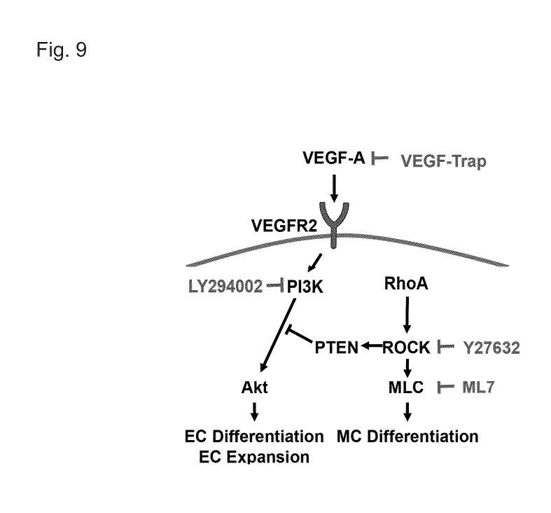

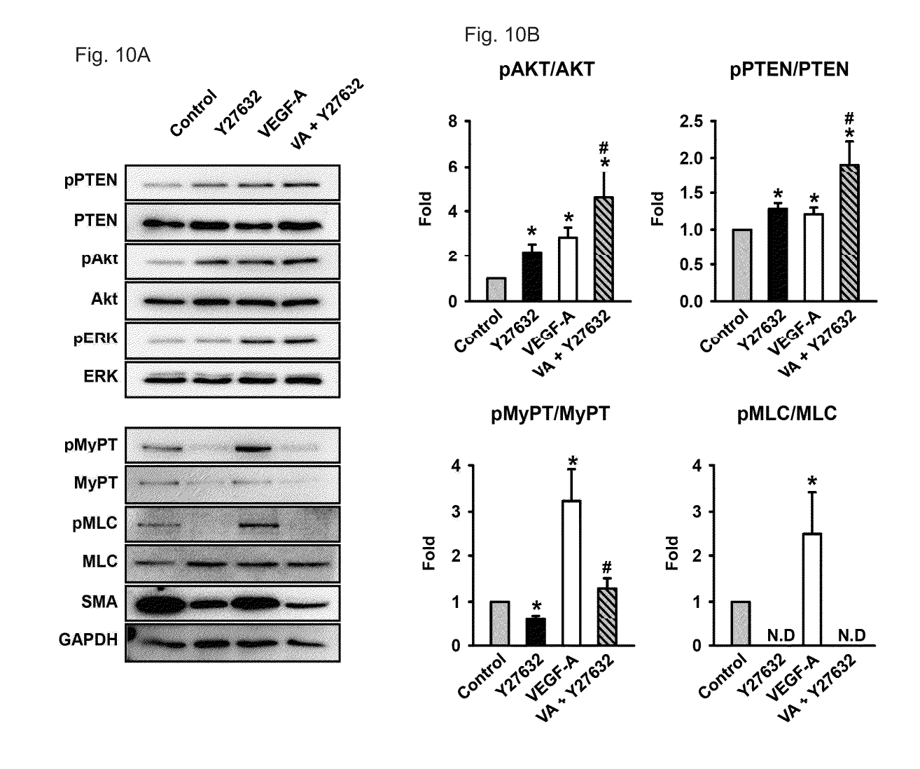

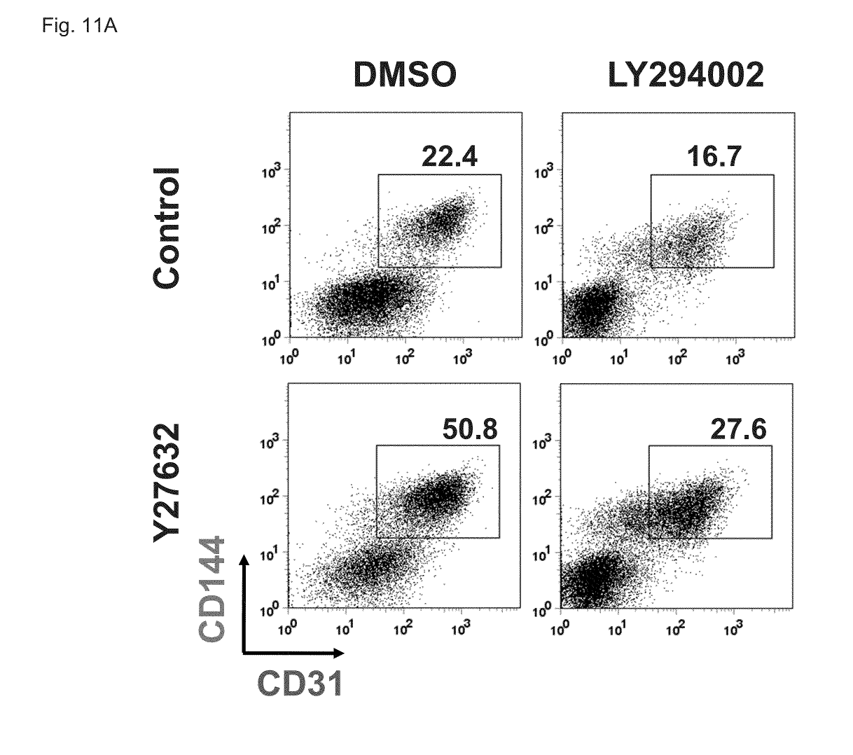

To investigate whether the ROCK suppression-induced EC expansion is VEGF-A dependent or not, we blocked the action of VEGF-A through pretreatment of VEGF-Trap during the differentiation (FIGS. 8A and 8B). VEGF-Trap totally abolished the ROCK suppression-induced expansion of ECs, indicating that the process is indeed VEGF-A dependent. Notably, ROCK suppression profoundly reduced ┌ SMA+MC populations whether or not VEGF-A was supplemented or blocked (FIGS. 8A and 8C). In other words, ROCK suppression alone significantly inhibits MC differentiation, and the combination of ROCK suppression and VEGF-A stimulation is able to synergistically promote the differentiation and expansion of ECs (FIG. 9). These data suggest that ROCK downstream signaling pathway responsible for EC differentiation could be separate from that for MC differentiation.

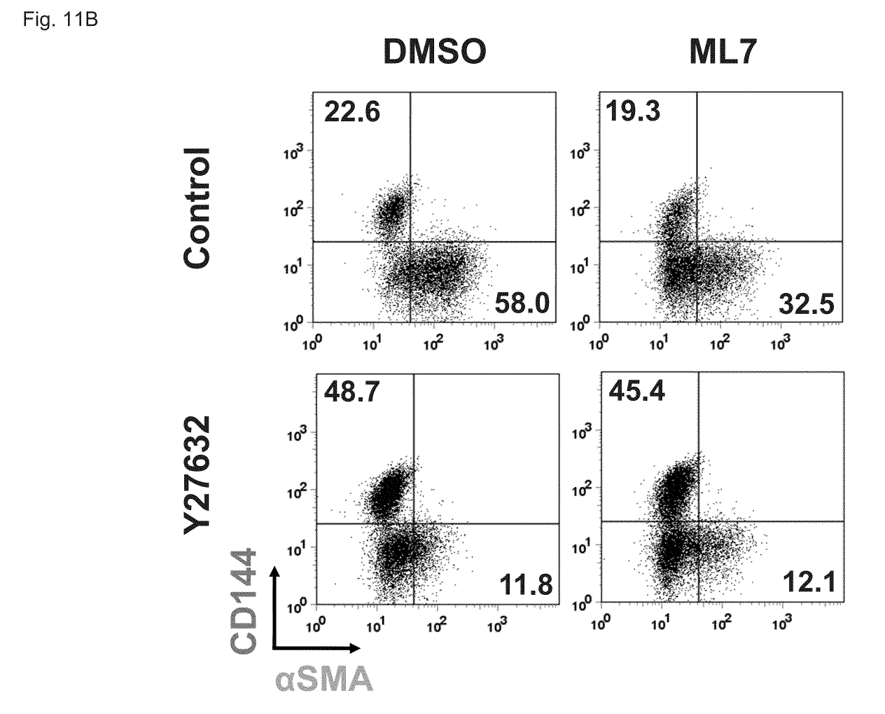

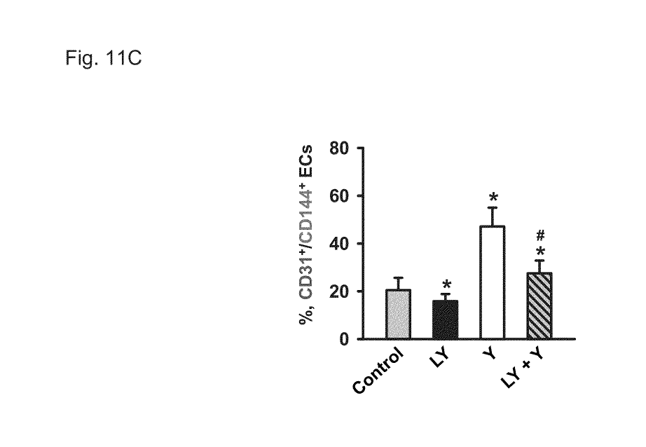

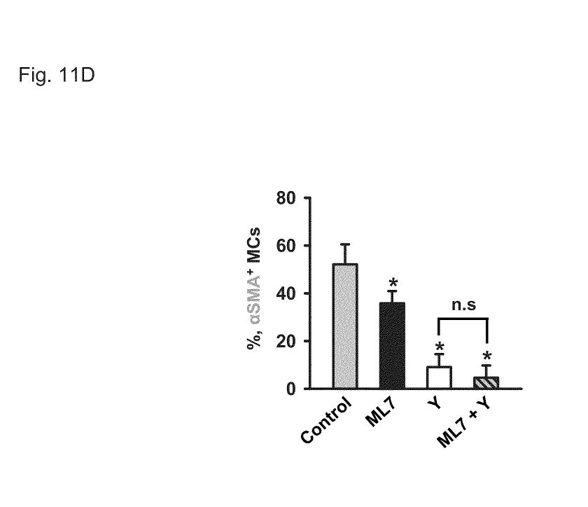

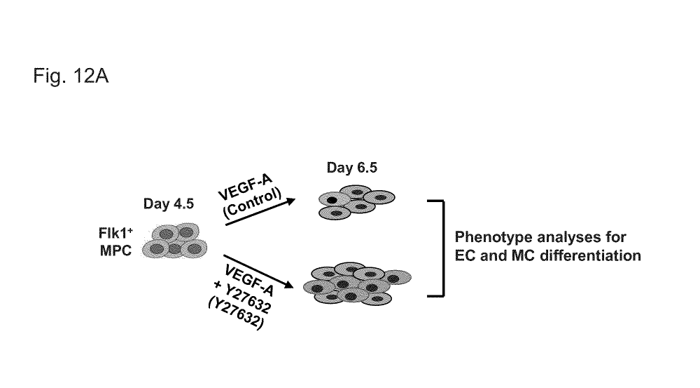

RhoA-ROCK signaling is known to stimulate the phosphatase activity of PTEN, leading to suppression of Akt activation in leukocytes and human embryonic kidney cells [43]. In agreement with this phenomenon, ROCK suppression via Y27632 phosphorylated PTEN (Ser380, Thr382 and Thr383) and Akt (Ser473) in the Flk1+ MPCs, but not ERK (FIGS. 10A and 10B). In comparison, VEGF-A alone or VEGF-A plus Y27632 phosphorylated all PTEN, Akt, and ERK (FIGS. 10A and 10B). Accordingly, PI3-kinase inhibitor LY294002 almost completely suppressed the ROCK suppression-induced differentiation and expansion of ECs under VEGF-A stimulus (FIGS. 11A and 11B). This demonstrates that ROCK suppression-induced PTEN inhibition promotes VEGF-A-induced PI3-kinase-Akt signaling, leading to enhanced proliferation, differentiation, and expansion of ECs (FIG. 9).

MyPT and myosin light chain (MLC) are crucial elements for myogenic differentiation, both of which are regulated by ROCK [23-25]. Indeed, ROCK suppression via Y27632 almost completely reduced the phosphorylation of MyPT and MLC, and in parallel reduced the protein levels of ┌ SMA regardless of VEGF-A stimulation in Flk1+ MPCs (FIGS. 10A and 10B). In fact, Y27632 reduced ┌ SMA+MC population by ˜83% and MLC kinase inhibitor ML7 (5┌ Mol) reduced ┌ SMA+MC population by ˜31%, and combination of Y27632 and ML7 further reduced ┌ SMA+MC population by ˜91% (FIGS. 11C and 11D). This collectively shows that ROCK suppression reduces the differentiation of MCs from Flk1+MPCs through inhibition of MyPT-MLC signaling pathway (FIG. 9).

### Example 2.4

### ROCK Suppression-Induced ECs Express Typical EC Markers and EC-Related Genes

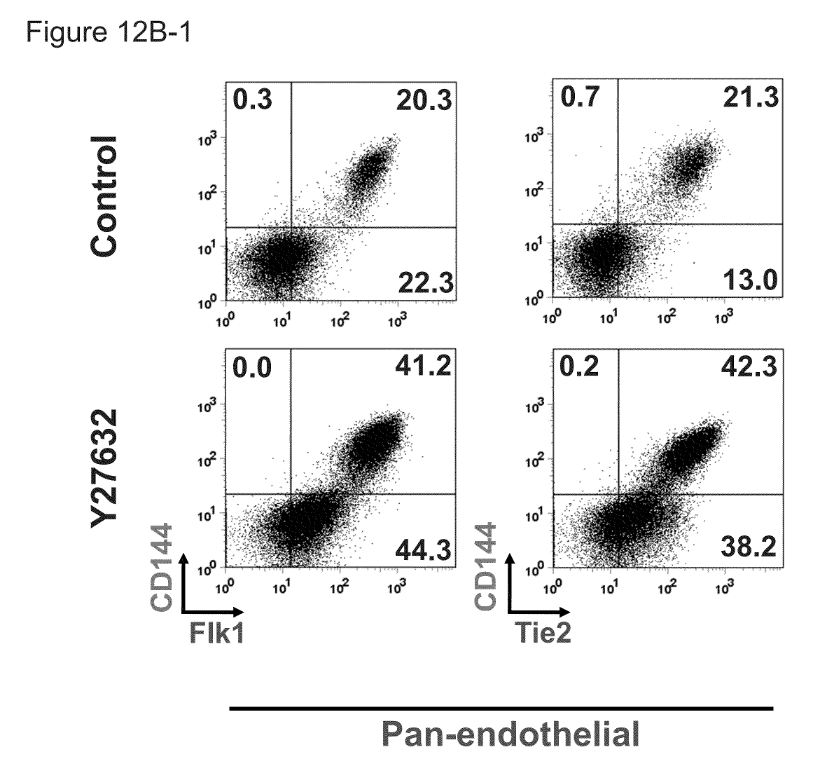

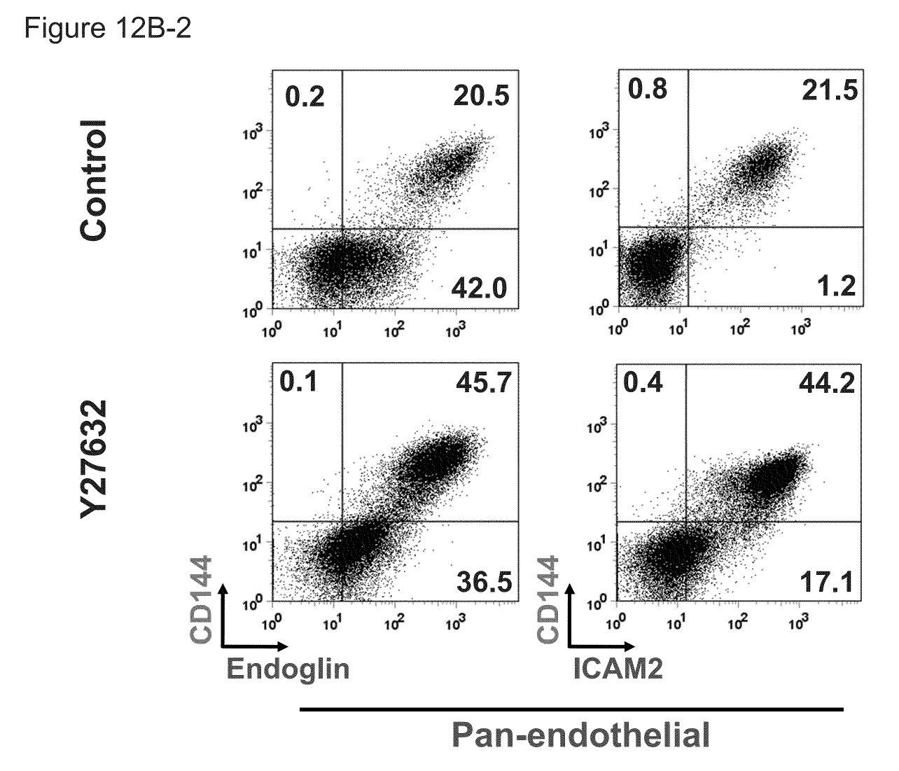

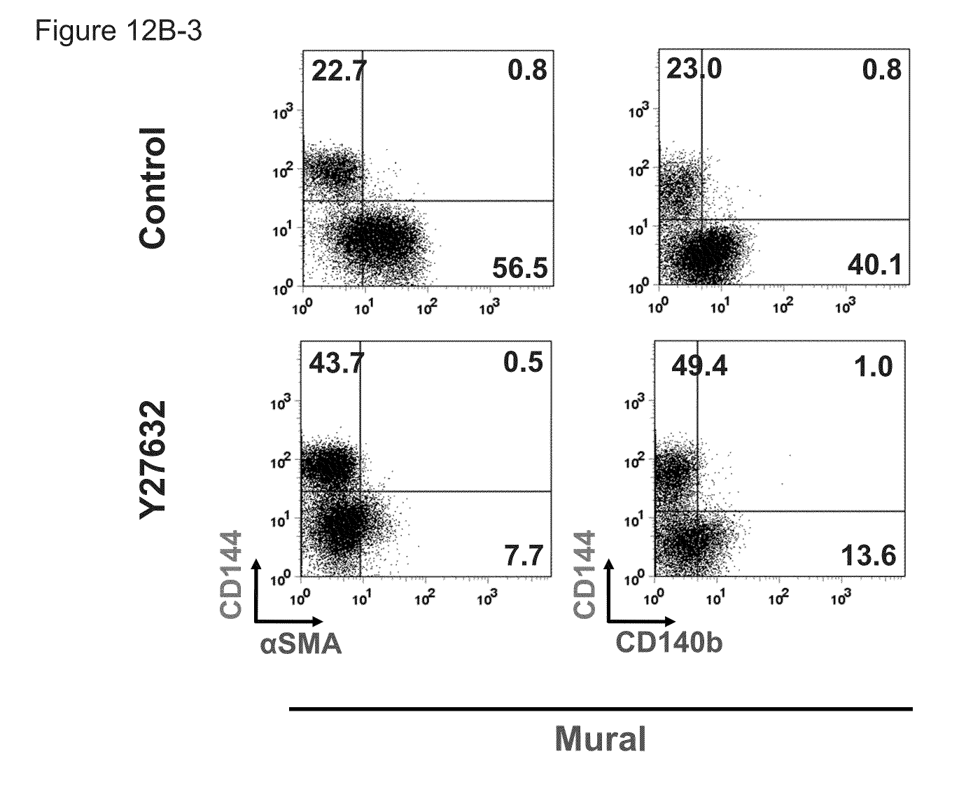

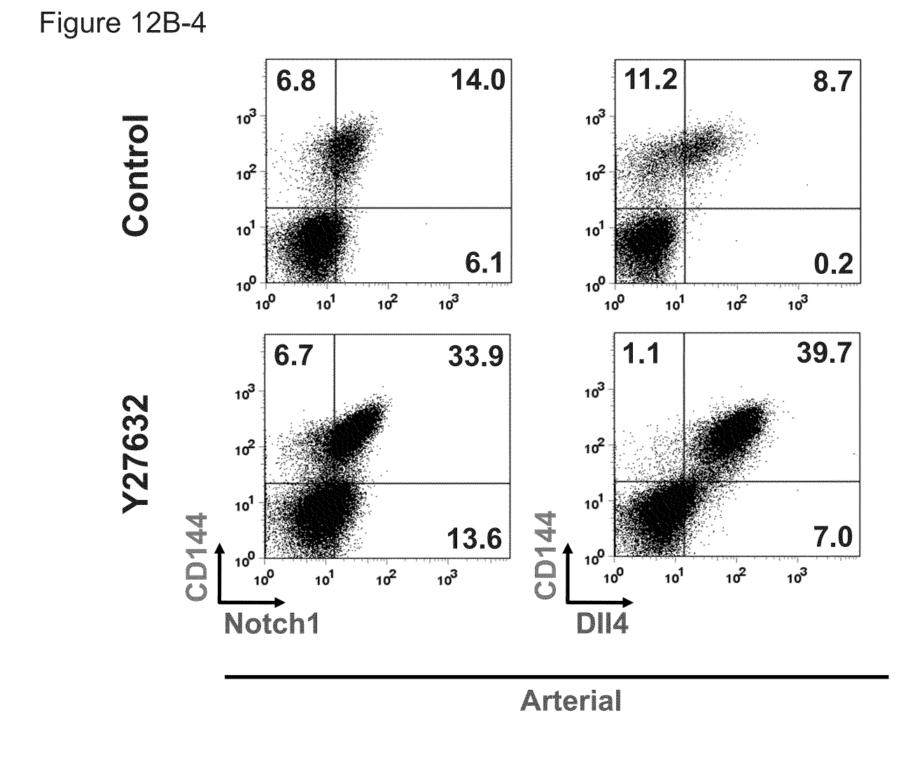

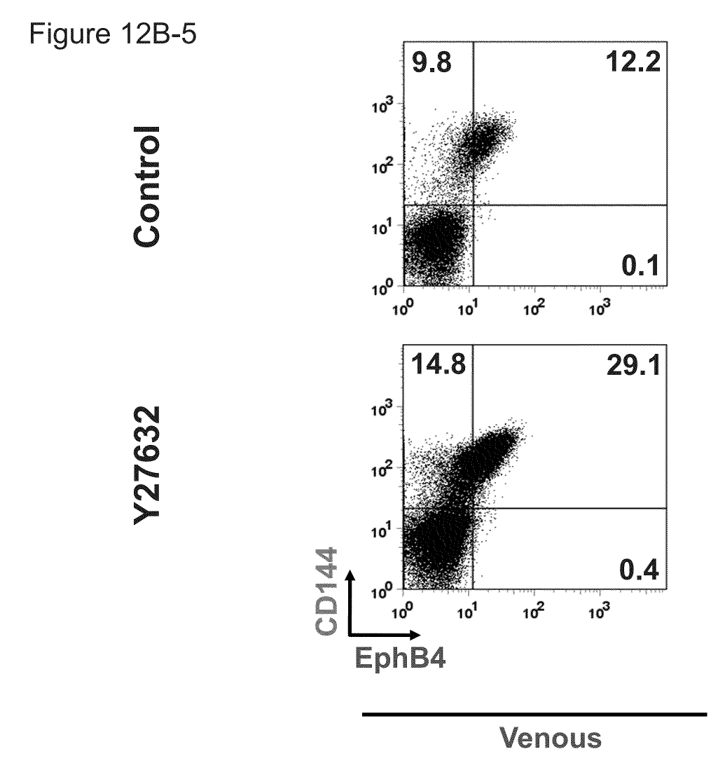

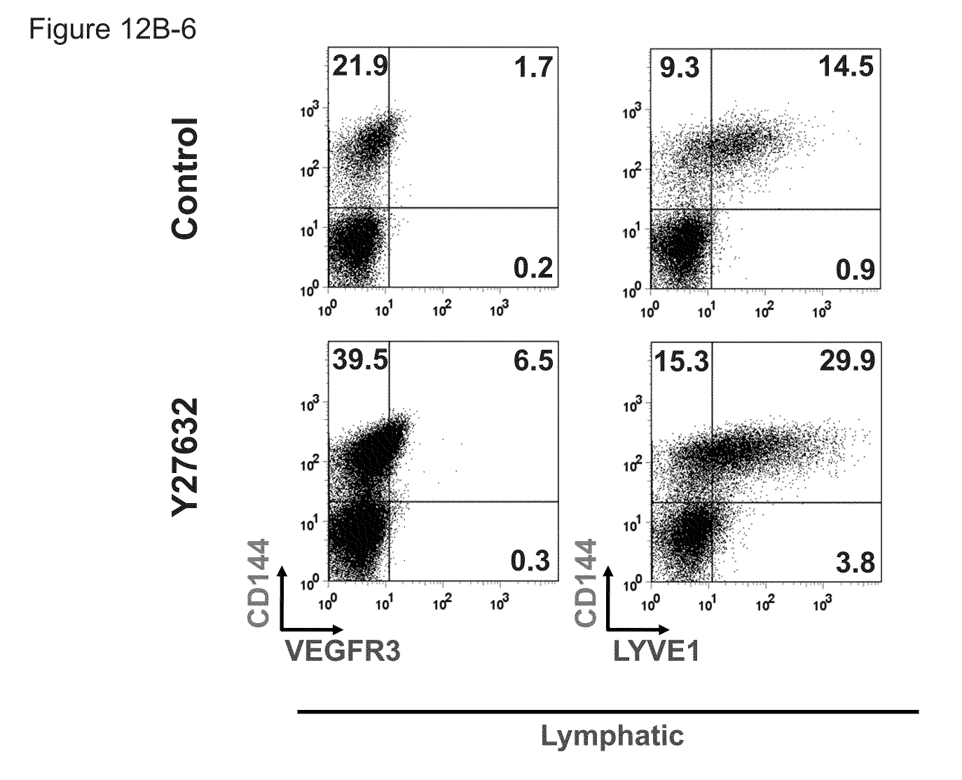

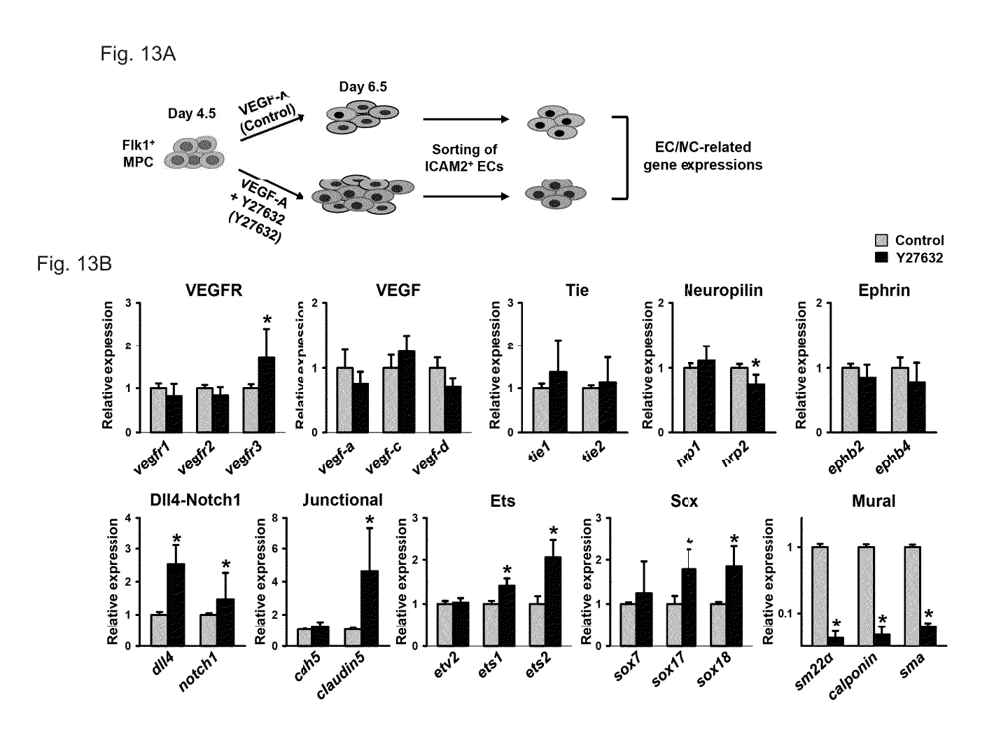

To characterize the ECs differentiated and expanded through ROCK suppression, we performed phenotypic analyses of various EC and MC markers at day 6.5 using FACS (FIG. 12A). Compared to control, Y27632 increased the population of CD144+ ECs expressing pan-endothelial cell surface markers (Flk1, Tie2, endoglin and ICAM2) by ˜2.0-fold, while it markedly reduced the populations of cells expressing ┌ SMA and CD140b (FIG. 12B). Further analyses revealed that Y27632 increased the population of CD144+ ECs that express Notch1 (arterial, 2.4-fold), D114 (arterial, 4.6-fold), EphB4 (venous, 2.4-fold), VEGFR3 (lymphatic, 3.8-fold), and LYVE-1 (lymphatic, 2.1-fold) (FIG. 12B). At day 6.5, most CD144+ cells co-expressed intercellular adhesion molecule-2 (ICAM2), a surface marker for ECs that is able to remain in its intact form after dissociation of cultured cells by trypsin-EDTA treatment. Taking the advantage of remaining surface expression of ICAM2 [44], we were able to isolate ICAM2+ ECs by MACS at a purity of ˜90% (FIG. 13A). We then further analyzed the mRNA expression of EC- and MC-related genes in the purified ICAM2+ cells (FIG. 13A). The ICAM2+ ECs induced by VEGF-A expressed typical EC-specific genes˜VEGF ligands, VEGF receptors, Tie receptors, neuropilins, ephrinB2-EphB4, D114-Notch1, EC-specific junctional proteins, and Ets and Sox family transcriptional factors, as well as MC-specific genes including SM22a, calponin, and SMA. Compared to the ICAM2+ ECs induced by VEGF-A, the ICAM2+ ECs induced by VEGF-A plus Y27632 expressed more vegfr3 (1.7-fold), dll4 (2.5-fold), notch1 (1.5-fold), claudin-5 (4.6-fold), ets1 (1.4-fold), ets2 (2.1-fold), sox17 (1.7-fold), and sox18 (1.8-fold), whereas they showed drastic reduced expression level of all SMC-specific genes—sm22a (25-fold), calponin (25-fold), and sma (17-fold) (FIG. 13B). Our interpretation of this finding is that the ICAM2+ ECs induced by VEGF-A plus Y27632 are further differentiated toward arterial ECs compared to those induced by VEGF-A only.

### Example 2.5

### ROCK Suppression Continuously Promotes the Expansion of ECs

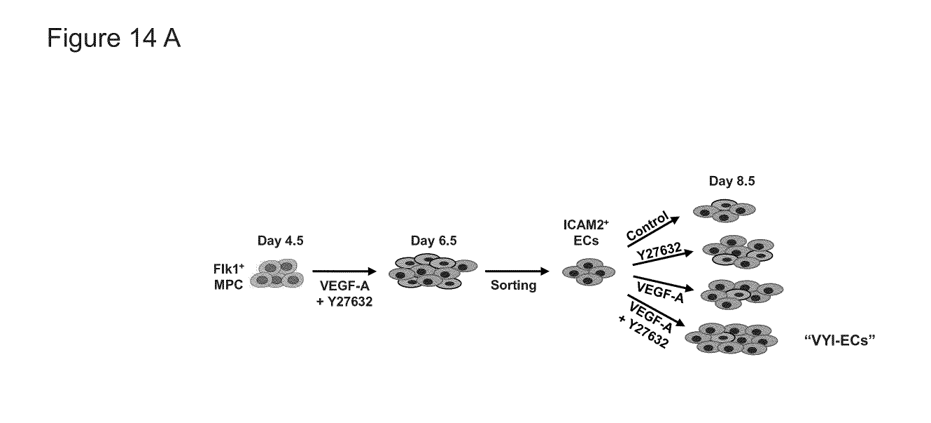

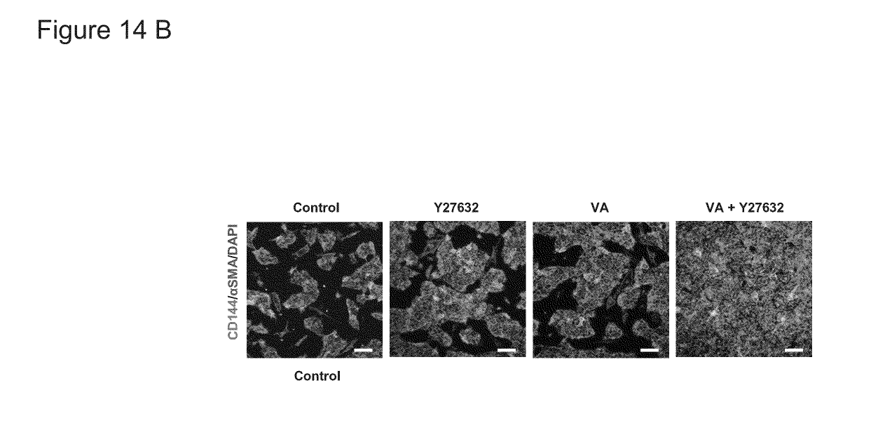

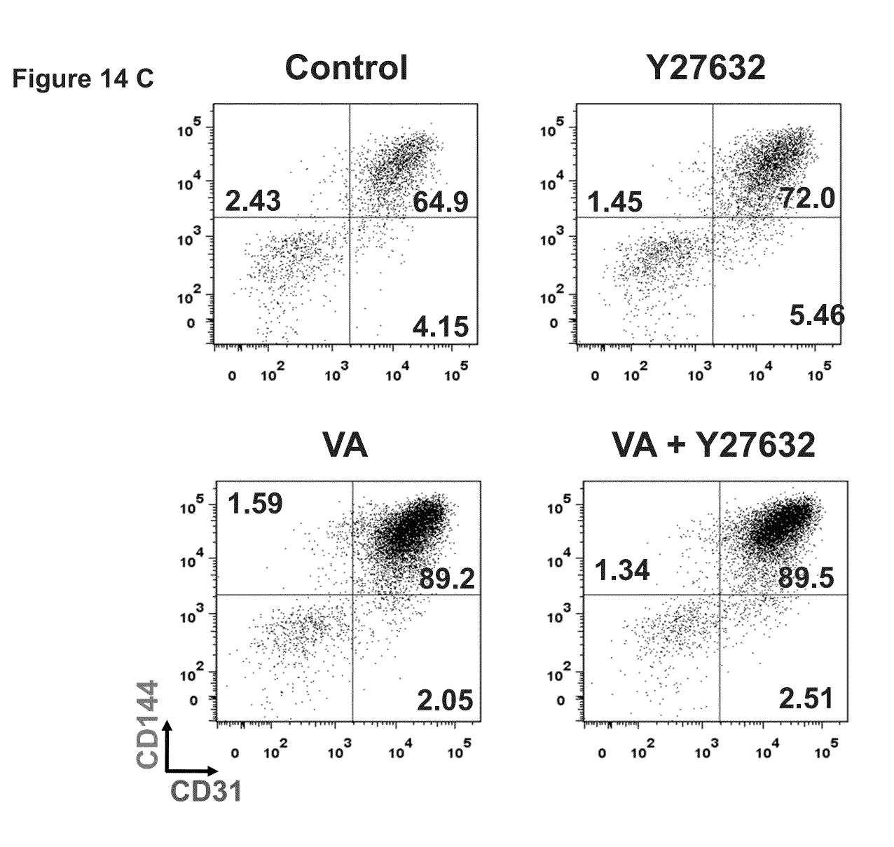

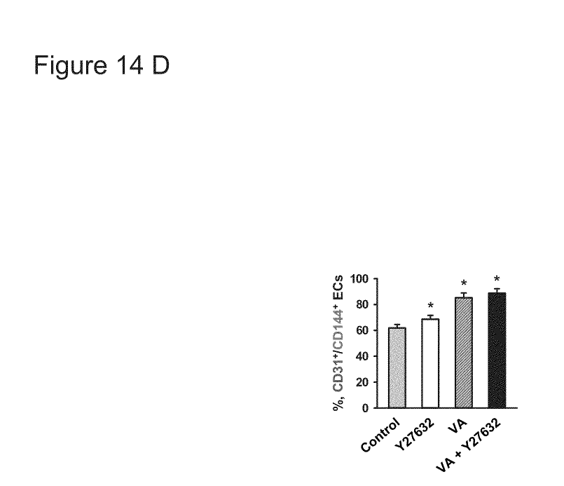

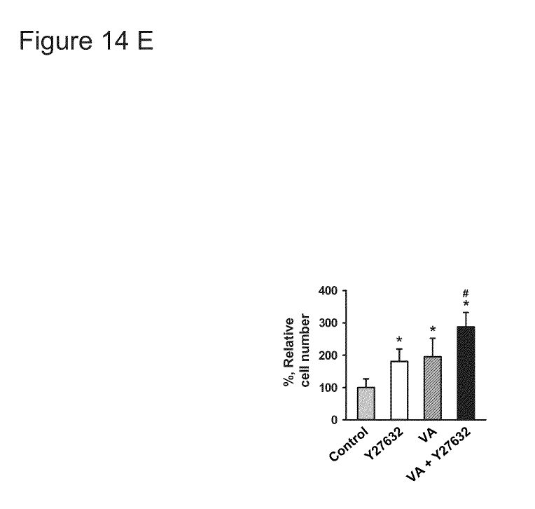

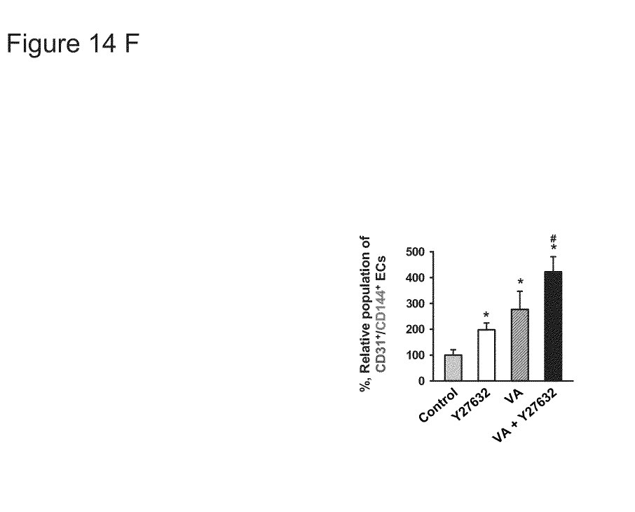

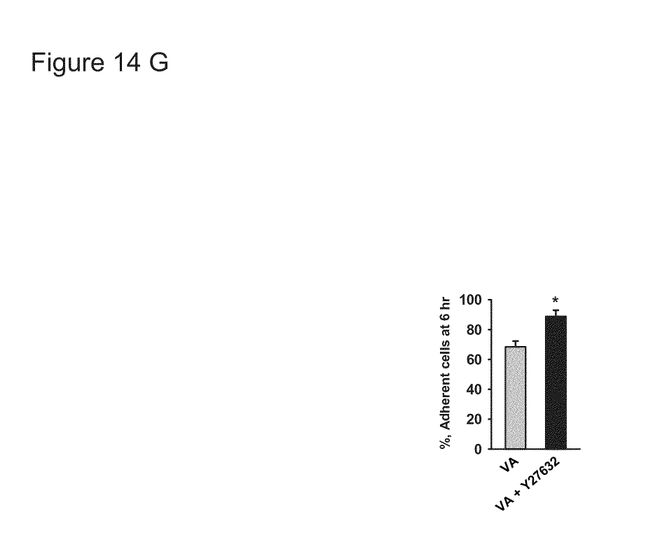

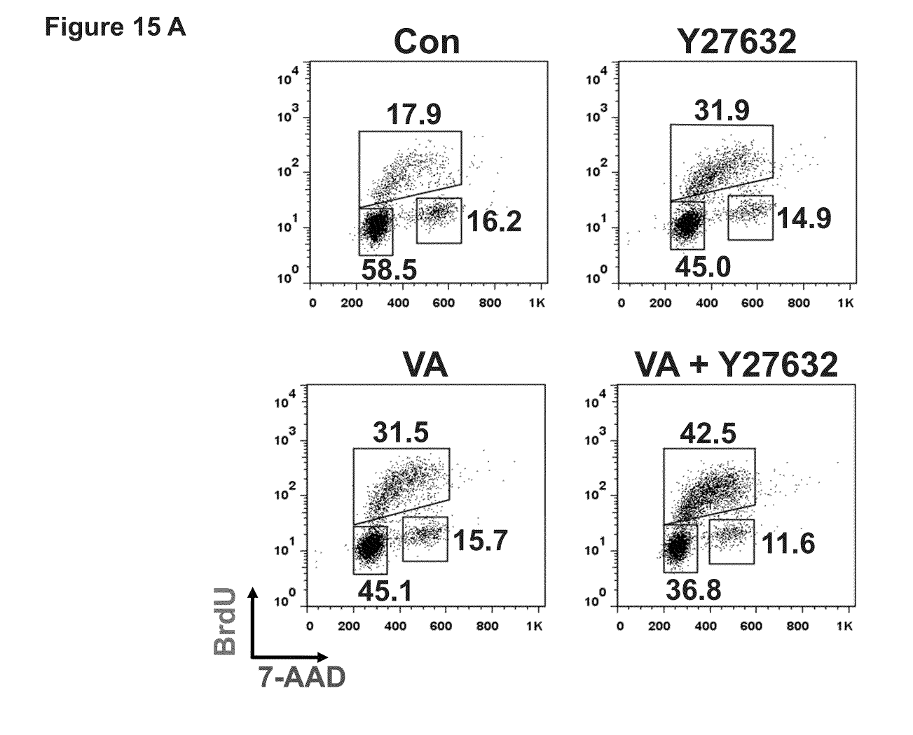

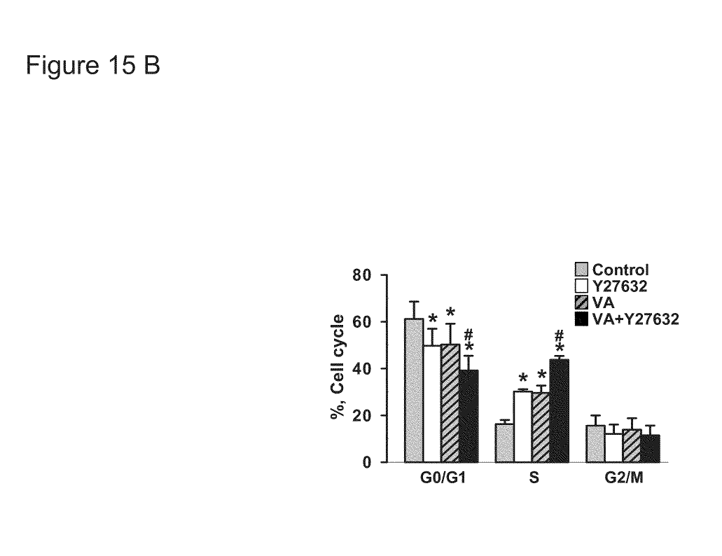

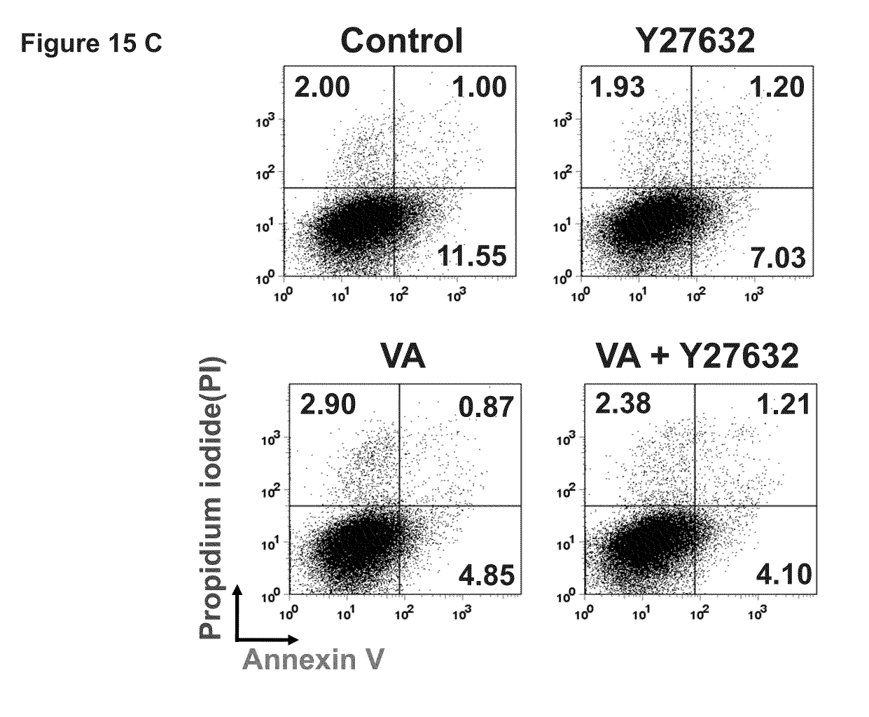

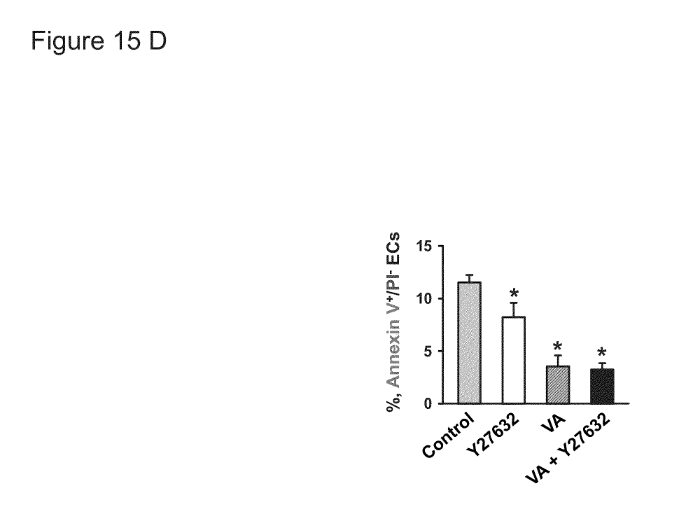

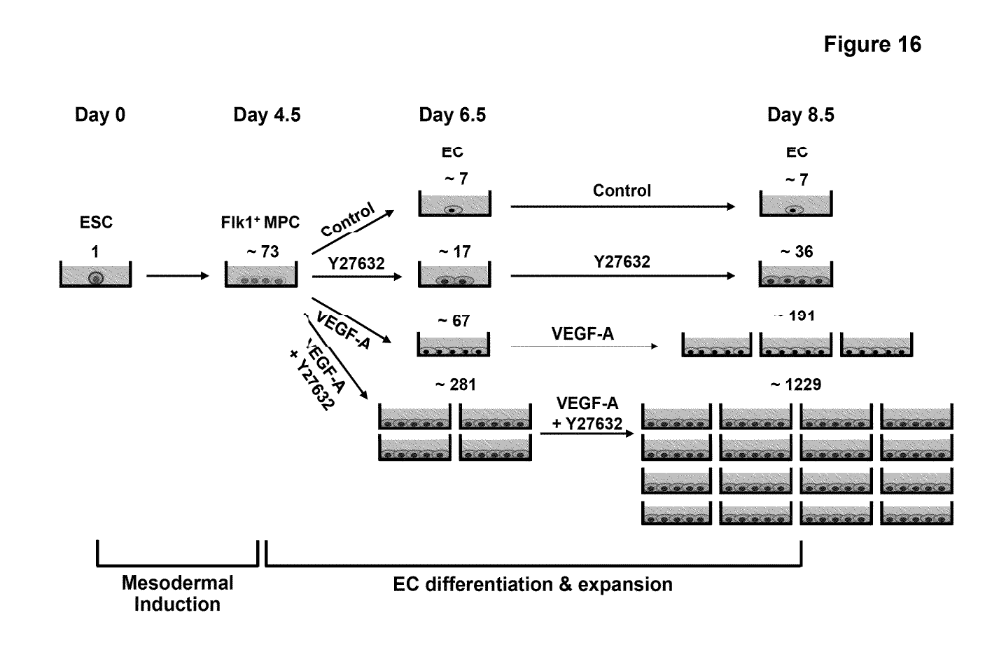

To maximize the amount of ECs obtained from ESC, we expanded the ICAM2+ ECs for 2 days using the same culture system with VEGF-A and Y27632 (FIG. 14A). When the ECs were grown for 2 days without either VEGF-A or Y27632, the population of CD31+/CD144+ ECs underwent a decrease of ˜30% from the initial population of ˜90% (FIG. 14B-D). This decrease in EC population was dramatically ameliorated by treatment of VEGF-A alone, in which case the CD31+/CD144+ population was only decreased by ˜5% instead of ˜30%. In contrast, treatment of Y27632 did not significantly affect the decrease of EC population: the EC population was decreased by ˜22% when Y27632 was treated alone, respectively (FIG. 14B-D). On the other hand, Y27632 alone or VEGF-A alone similarly increased the total cell number by ˜1.8-fold and ˜1.9-fold, respectively, and combination of Y27632 and VEGF-A further increased the total cell number by ˜2.9-fold (FIG. 14E). This way, we were further able to obtain ˜4.2-fold expansion of CD31+/CD144+ ECs from the ICAM2+ ECs (FIGS. 14B-F) In the presence of VEGF-A, Y27632 promoted the adhesion of ICAM2+ ECs onto the plate by 1.3-fold at 6 hrs after plating (FIG. 14G). In these purified ECs, Y27632 and VEGF-A each promoted the proliferation of ECs to a similar level, and combination of Y27632 and VEGF-A promoted EC proliferation to a greater degree. We also performed FACS analysis to examine the cell cycle of CD144+ ECs in ICAM2+ ECs at 12 hrs after plating, and found that, compared to control, Y27632 and VEGF-A each increased the percentage of the cells in S phase to a similar degree (˜1.8-fold), and that the combination of the two compounds further increased the percentage of proliferating cells by 2.7-fold (FIGS. 15A and 15B). Furthermore, we noted that Y27632 mildly inhibited the apoptosis of ECs, whereas VEGF-A strongly inhibited the same (FIGS. 15C and 15D). Based on these findings, we conclude that further expansion of high purity ECs (>90%) could be achieved from ESC by combination of simultaneous ROCK suppression and VEGF-stimulation—these high purity ECs are hereafter referred to as “VEGF-A plus Y27632-induced and anti-ICAM2 antibody-sorted ECs (VYI-ECs)” (FIG. 14A). We estimated how many VYI-ECs can be obtained at day 8.5 from one ESC using our 2D Matrigel system: from ˜73 Flk1+ MPCs differentiated from one ESC, only ˜36 ECs and ˜191 ECs were generated by treatment of either Y27632 or VEGF-A alone, respectively; in contrast, ˜1,229 ECs were generated by combination of Y27632 and VEGF-A (FIG. 16).

### Example 2.6

### VYI-ECs have Neovasculogenic Capabilities In Vitro and In Vivo

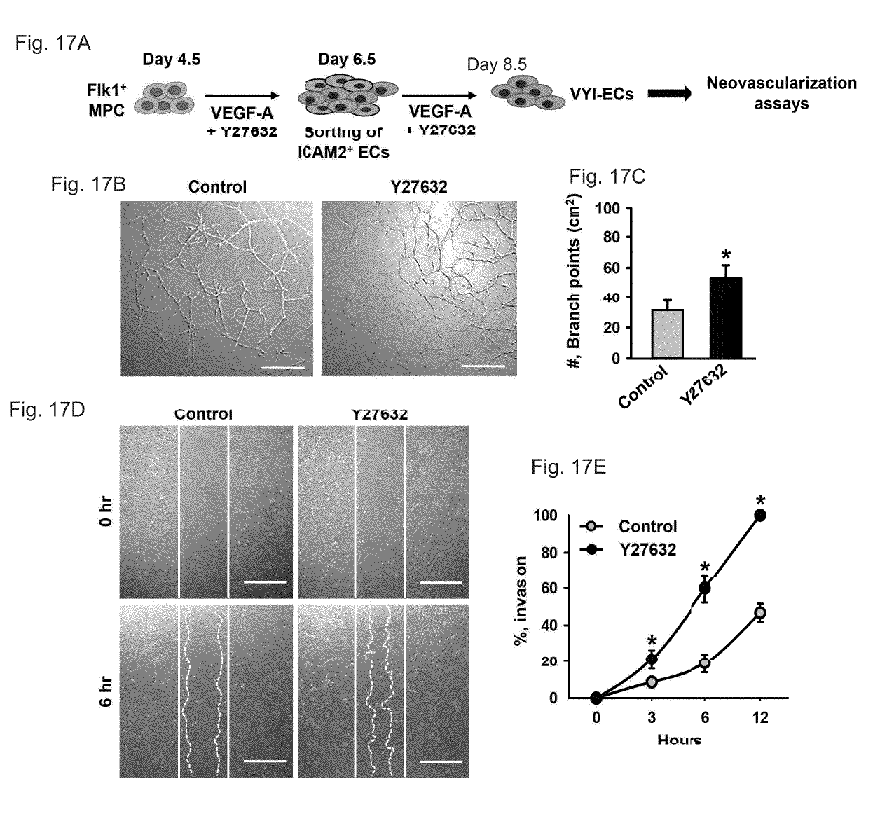

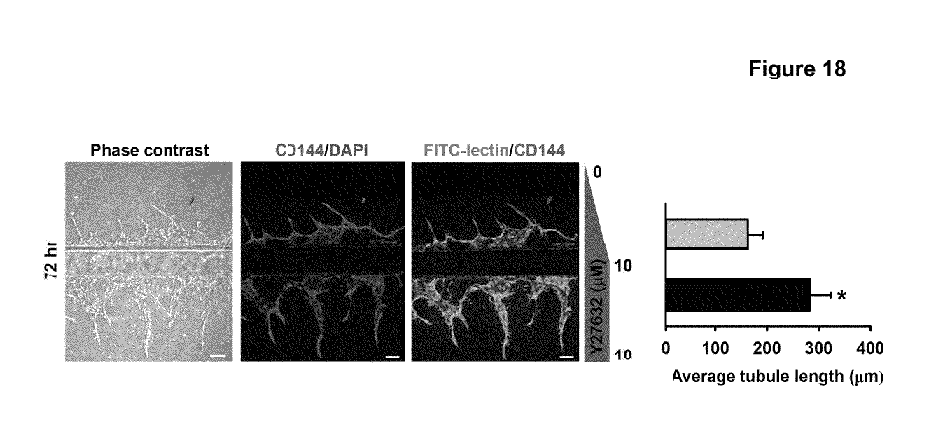

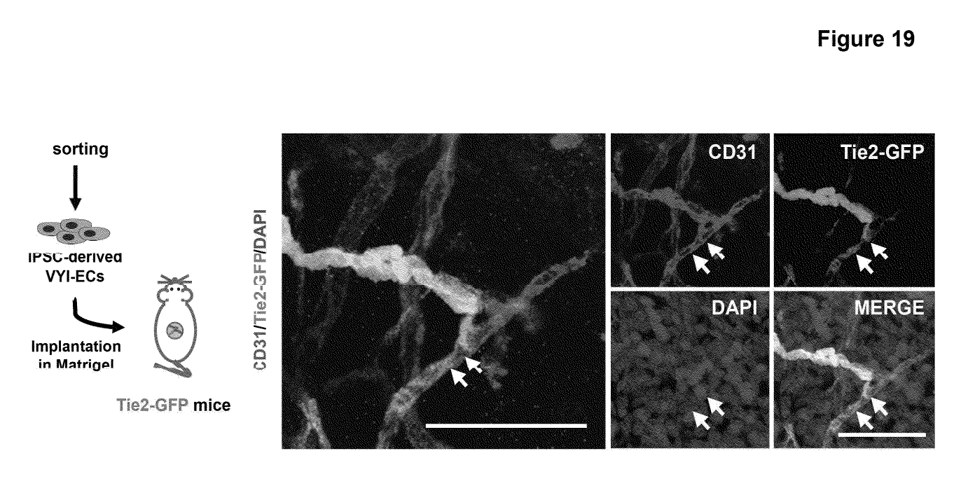

To determine the in vitro neovasculogenic properties of VYI-ECs, various assays including tube formation, scratch wound healing, and angiogenic ability using a microfluidic system were performed (FIG. 17A). Treatment of VEGF-A alone (Control) induced the formation of vascular network (˜31.3±6.4/cm², measured by counting the number of branch points) at 12 hr after VYI-ECs have been plated (FIG. 17B); VEGF-A also induced a gradual invasion of ECs into the wound region (˜46.7±4.9% at 12 hr) (FIG. 17D). Addition of Y27632 increased the formation of vascular networks (1.7-fold) as well as cell invasion into the wound region (2.1-fold) (FIGS. 17B-E). We also designed a “microfluidic angiogenesis assay system” that allowed us to assess the angiogenic property of VYI-ECs, and observed that co-treatment of Y27632 increased the length of sprouting CD144+ VYI-ECs by 1.7-fold at 72 hr (FIG. 18) [38]. In vivo neovasculogenic potential of VYI-ECs derived from iPSCs (FVB origin) was tested using a Matrigel plug implantation into Tie2-GFP+ mice (FVB origin) [45], of which the blood vessels and a subset of hematopoietic cells of the recipient mouse are labeled with GFP in order to distinguish the donor-derived vessels from recipient-derived vessels. Immunofluorescent staining showed both recipient-derived GFP+/CD31+ vessels and the implanted VYI-EC-derived GFP−/CD31+ vessels in the Matrigel; noticeably, some of the VYI-EC-derived vessels were connected to the adjacent recipient blood vessels (FIG. 19), suggesting that VYI-ECs were successfully incorporated with the recipient circulatory network. Based on these observations, we conclude that VYI-ECs have neovasculogenic capabilities in vivo as well as in vitro.

### Example 2.7

### CsA Promotes the Differentiation and Expansion of CMs from Flk1+MPCs

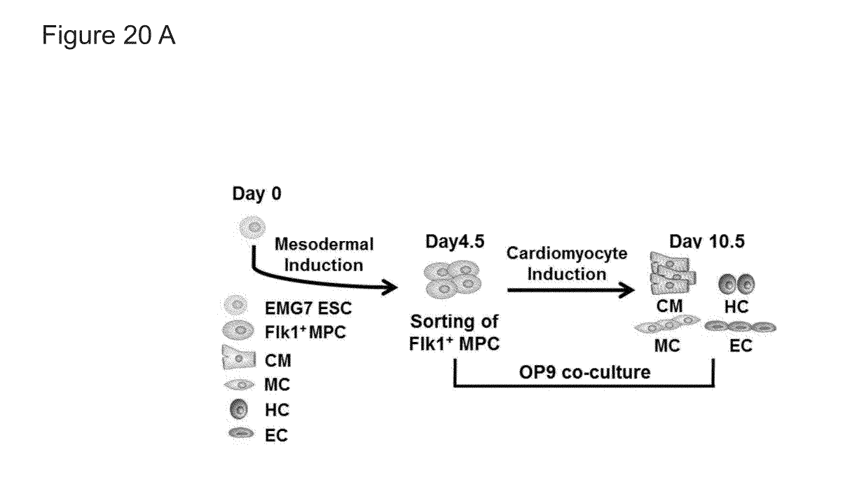

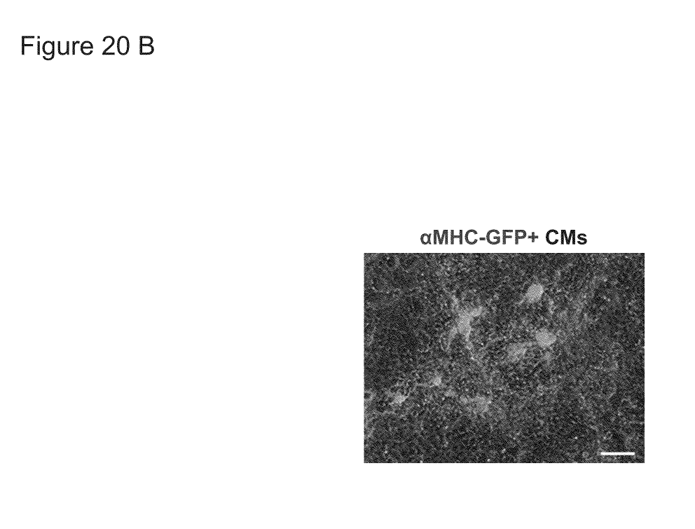

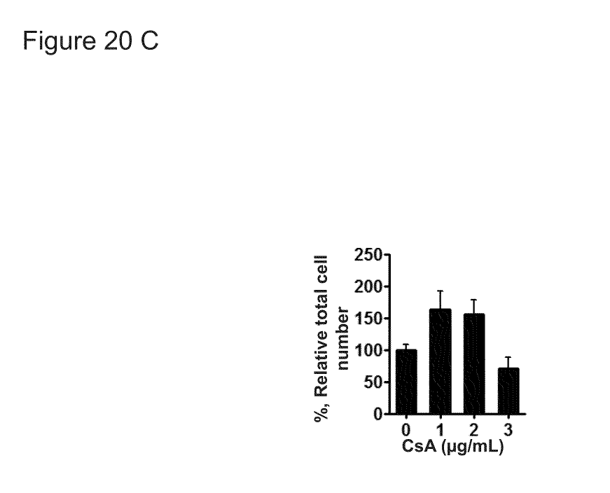

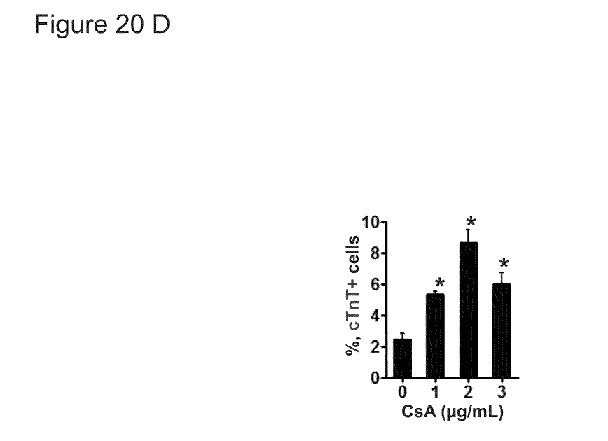

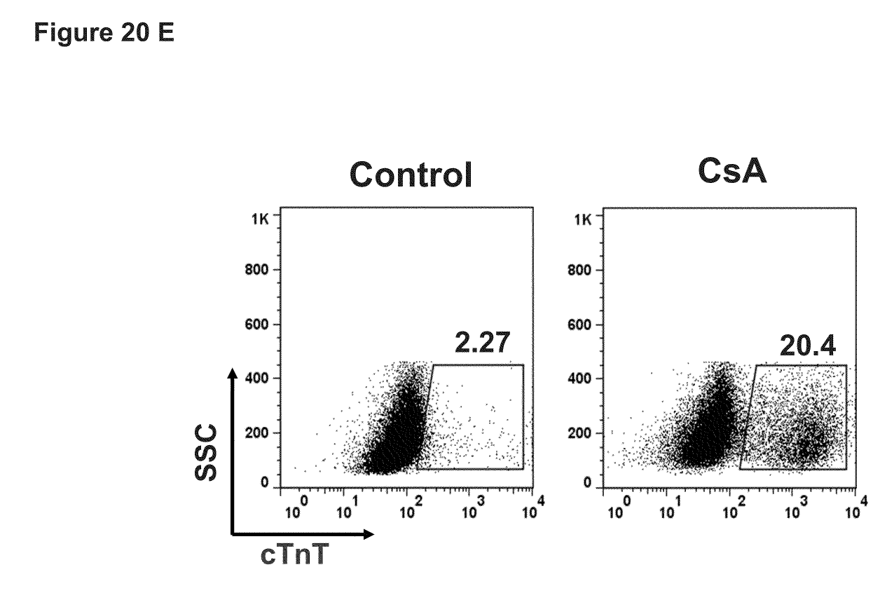

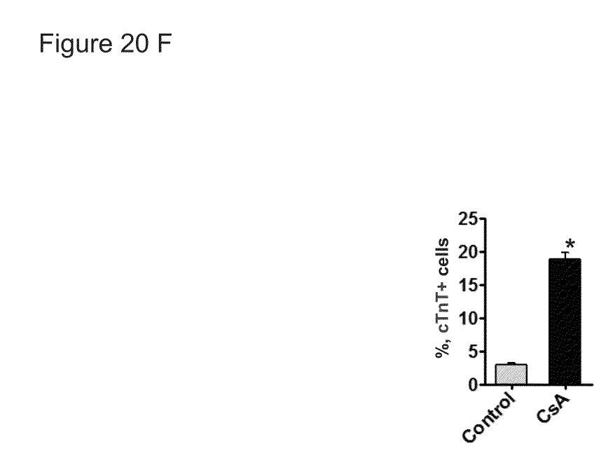

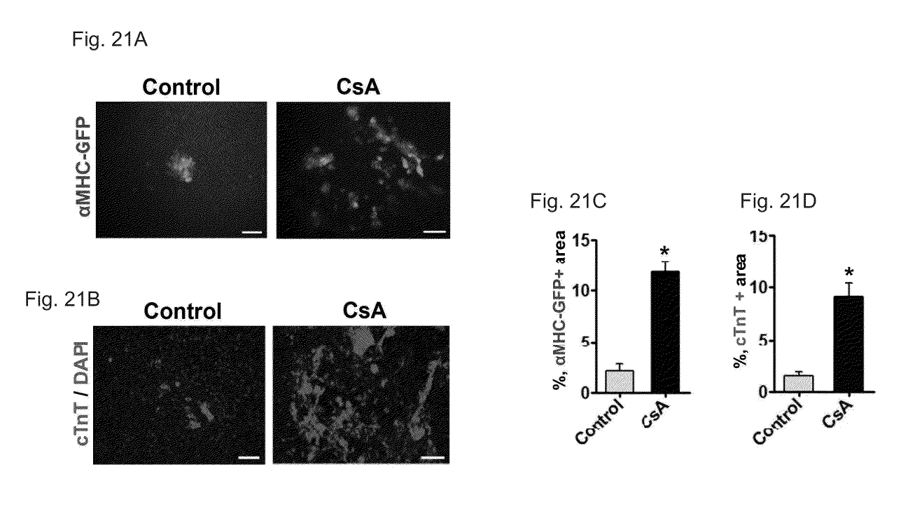

EMG7 ESC cell line which has cardiac specific alpha-myosin heavy chain (┌ MHC) promoter driven enhanced GFP gene was obtained from Prof. Jun K. Yamashita in Kyoto University (Japan) and was used for monitoring CM differentiation. Accordingly, GFP has been detected in the differentiating CM. To support CM differentiation, OP9 co-culture system was used. Flk1+ MPCs were differentiated from EMG7 ESC, and when the Flk1+ MPCs were purified at day 4.5 and cultured on OP9 cells in the differentiation medium (FIG. 20A). At day 8.5-10.5, self-beating and GFP-expressing CMs were detected (FIG. 20B). Consistent with previous reports [36,37], CsA increased not only the percentage of cTnT+CM population, but also the total cell number in dose-dependent manner (FIGS. 20C and 20D). Among them, 2┌ g/mL of CsA increased ˜6.5-fold cTnT+ CMs (FIGS. 20E and 20F). Therefore, for following experiments, 2┌ g/mL of CsA (hereafter referred to as CsA) was chosen to use based on its maximal effect on differentiation and expansion of CMs from Flk1+ MPCs (FIGS. 20C and 20D). Live cell images and immunofluorescence staining on day 10.5 showed that CsA increased the area of ┌ MHC GFP+ (˜5.7-fold) and cTnT+ (˜6.2-fold) CMs (FIGS. 21A-D).

### Example 2.8

### ROCK Suppression with CsA Promotes the Differentiation and Expansion of CMs from Flk1+ MPCs

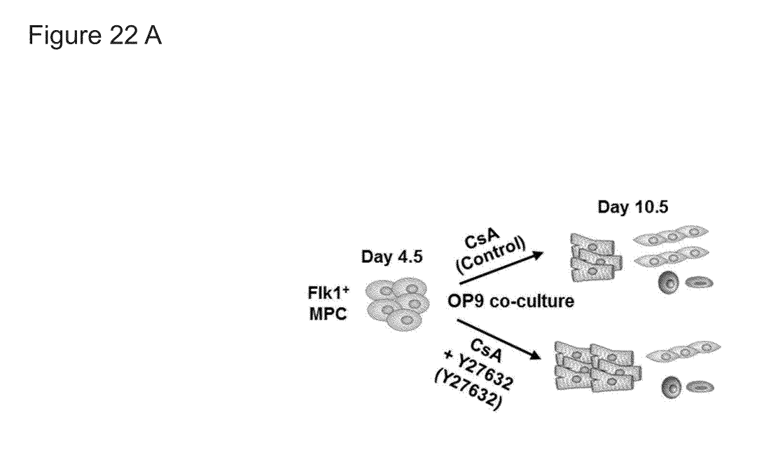

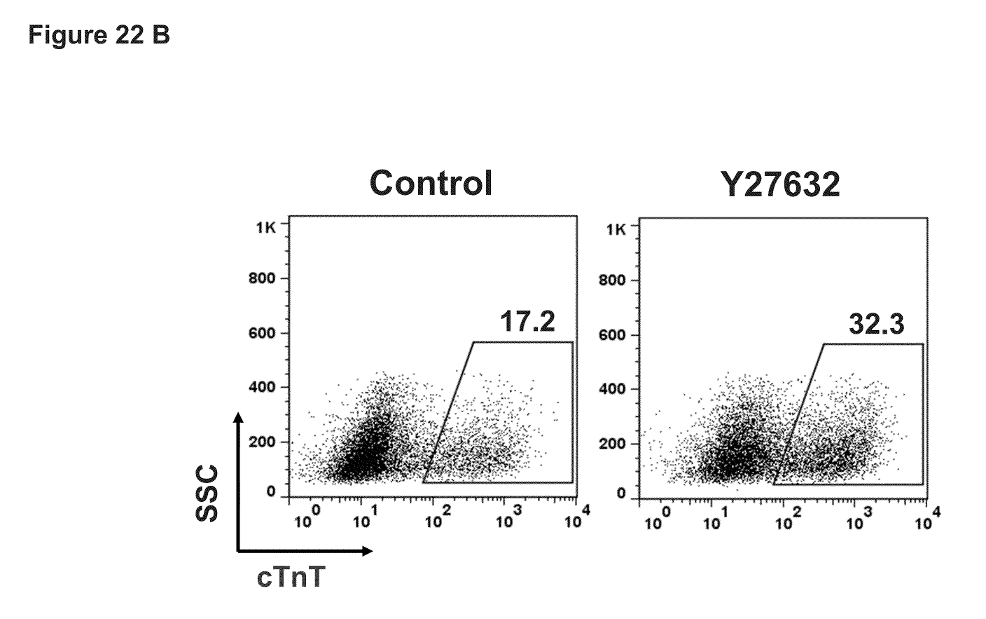

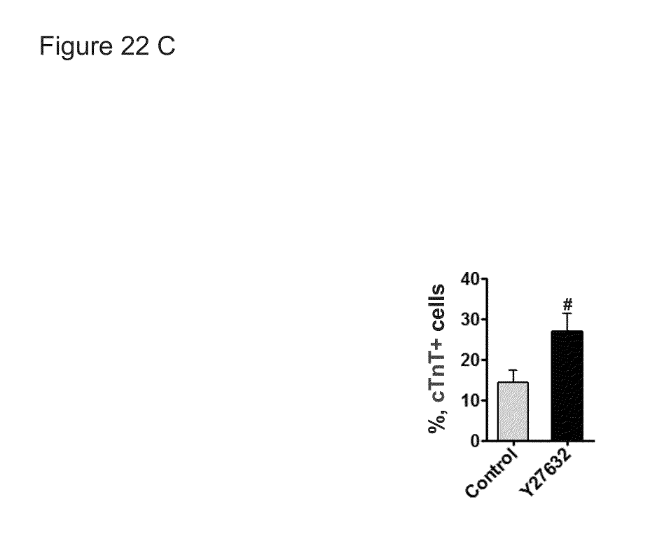

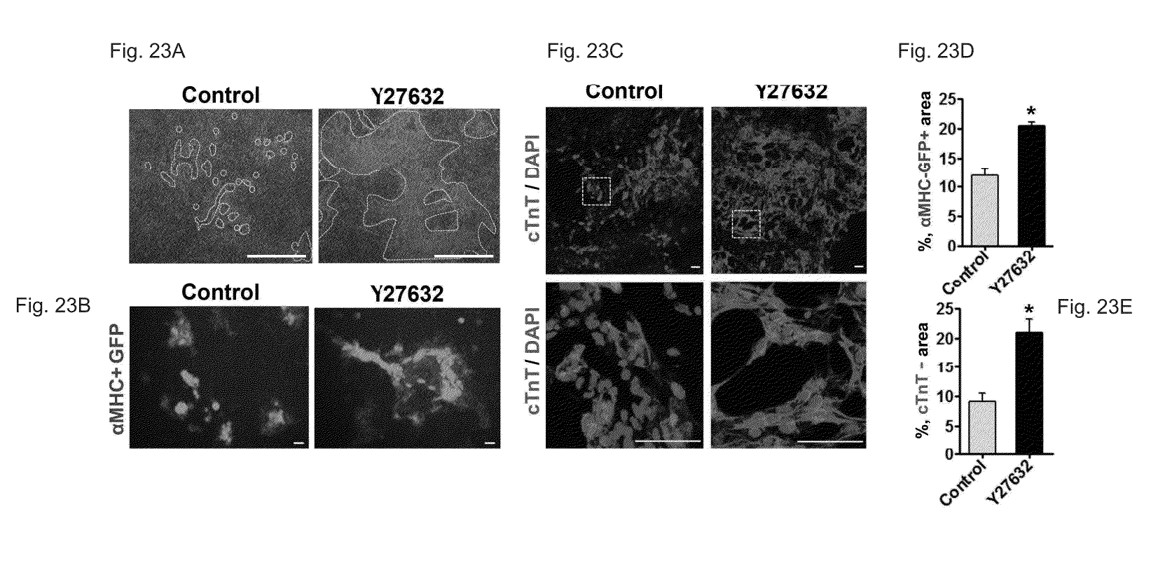

Previous reports [46,47] demonstrated that Rho-ROCK signaling pathway modulated the differentiation of MCs. To examine role of ROCK, addition of Y27632 was performed during the CsA-induced CM differentiation in the OP9 co-culture system (FIG. 22A). Surprisingly, the addition of Y27632 increased cTnT+ CMs ˜1.9-fold compared to control CsA only (FIGS. 22B and 22C). In addition, the area of self-beating CMs was strikingly increased after addition of Y27632 (FIG. 23A). Live cell images and immunofluorescence staining on day 10.5 showed that addition of Y27632 significantly increased the area of ┌ MHC GFP+(˜1.7-fold) and cTnT+ CMs (˜2.3-fold) (FIGS. 23B-E).

### Example 2.9

### ROCK Suppression Increased the Number of CMs in the Feeder-Free Condition

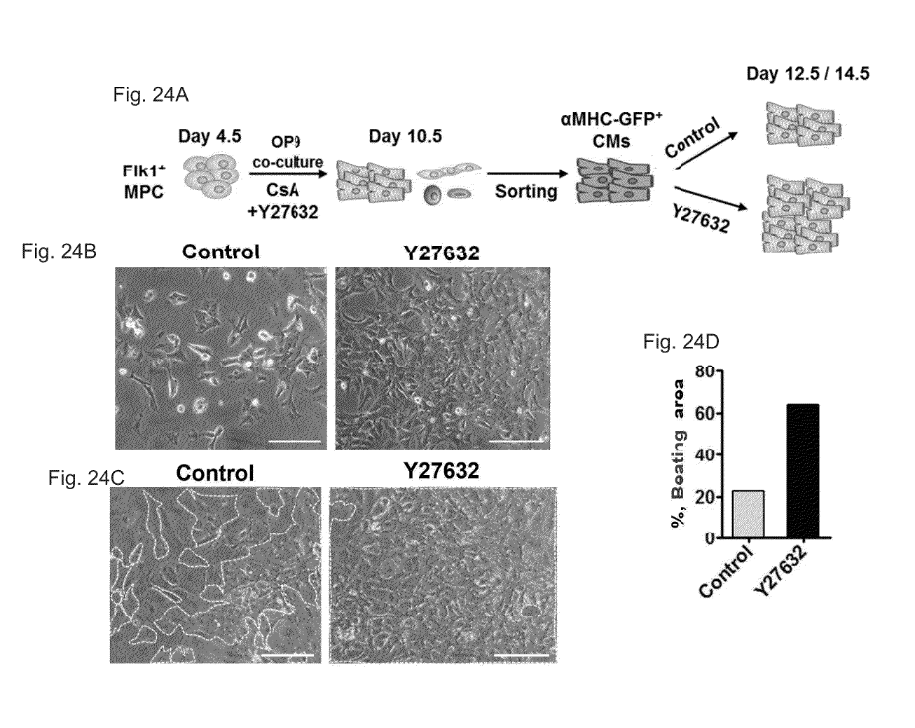

To avoid cell contamination from using feeder cells for differentiation of CMs, we sorted out ┌ MHC GFP+CMs at day 10.5 using FACS and plated on to 0.1% gelatin coated dish without feeder cells in the differentiation medium with or without Y27362 (FIG. 24A). Although we plated same number of cells, much more self-beating CMs were observed in the Y27632-addition group compared to control at day 12.5 (FIG. 24B). The area of self-beating CMs was continuously more (˜2.8-fold compared control) observed in the Y27632-addition group after confluent state at day 14.5 (FIGS. 24C and 24D).

### EXAMPLES

1. Yamashita J K. ES and iPS cell research for cardiovascular regeneration. Exp Cell Res. 2010; 316(16):2555-2559.
2. Nishikawa S I, Nishikawa S, Hirashima M, Matsuyoshi N, Kodama H. Progressive lineage analysis by cell sorting and culture identifies FLK1+ VE-cadherin+ cells at a diverging point of endothelial and hemopoietic lineages. Development. 1998; 125(9):1747-1757.
3. Hirashima M, Kataoka H, Nishikawa S, Matsuyoshi N, Nishikawa S. Maturation of embryonic stem cells into endothelial cells in an in vitro model of vasculogenesis. Blood. 1999; 93(4):1253-1263.
4. Yamashita J, Itoh H, Hirashima M, et al. Flk1-positive cells derived from embryonic stem cells serve as vascular progenitors. Nature. 2000; 408(6808):92-96.
5. Yamashita J K. Differentiation of arterial, venous, and lymphatic endothelial cells from vascular progenitors. Trends Cardiovasc Med. 2007; 17(2):59-63.
6. Narazaki G, Uosaki H, Teranishi M, et al. Directed and systematic differentiation of cardiovascular cells from mouse induced pluripotent stem cells. Circulation. 2008; 118(5):498-506.
7. Yurugi-Kobayashi T, Itoh H, Schroeder T, et al. Adrenomedullin/cyclic AMP pathway induces Notch activation and differentiation of arterial endothelial cells from vascular progenitors. Arterioscler Thromb Vasc Biol. 2006; 26(9): 1977-1984.
8. Yamamizu K, Kawasaki K, Katayama S, Watabe T, Yamashita J K. Enhancement of vascular progenitor potential by protein kinase A through dual induction of Flk-1 and Neuropilin-1. Blood. 2009; 114(17):3707-3716.
9. Yamamizu K, Furuta S, Katayama S, et al. The kappa opioid system regulates endothelial cell differentiation and pathfinding in vascular development. Blood. 2011; 118(3):775-785.
10. Kawasaki K, Watabe T, Sase H, et al. Ras signaling directs endothelial specification of VEGFR2+ vascular progenitor cells. J Cell Biol. 2008; 181(1):131-141.
11. Bekhite M M, Finkensieper A, Binas S, et al. VEGF-mediated PI3K class I A and PKC signaling in cardiomyogenesis and vasculogenesis of mouse embryonic stem cells. J Cell Sci. 2011; 124(Pt 11):1819-1830.
12. Joo H J, Kim H, Park S W, et al. Angiopoietin-1 promotes endothelial differentiation from embryonic stem cells and induced pluripotent stem cells. Blood. 2011; 118(8):2094-2104.
13. Watabe T, Nishihara A, Mishima K, et al. TGF-beta receptor kinase inhibitor enhances growth and integrity of embryonic stem cell-derived endothelial cells. J Cell Biol. 2003; 163(6):1303-1311.
14. James D, Nam H S, Seandel M, et al. Expansion and maintenance of human embryonic stem cell-derived endothelial cells by TGFbeta inhibition is Id1 dependent. Nat Biotechnol. 2010; 28(2):161-166.
15. Noghero A, Bussolino F, Gualandris A. Role of the microenvironment in the specification of endothelial progenitors derived from embryonic stem cells. Microvasc Res. 2010; 79(3):178-183.
16. Wang Z Z, Au P, Chen T, et al. Endothelial cells derived from human embryonic stem cells form durable blood vessels in vivo. Nat Biotechnol. 2007; 25(3):317-318.
17. Coultas L, Chawengsaksophak K, Rossant J. Endothelial cells and VEGF in vascular development. Nature. 2005; 438(7070):937-945.
18. Lohela M, Bry M, Tammela T, Alitalo K. VEGFs and receptors involved in angiogenesis versus lymphangiogenesis. Curr Opin Cell Biol. 2009; 21(2): 154-165.
19. Riento K, Ridley A J. Rocks: multifunctional kinases in cell behaviour. Nat Rev Mol Cell Biol. 2003; 4(6):446-456.
20. McBeath R, Pirone D M, Nelson C M, Bhadriraju K, Chen C S. Cell shape, cytoskeletal tension, and RhoA regulate stem cell lineage commitment. Dev Cell. 2004; 6(4):483-495.
21. Jaffe A B, Hall A. Rho GTPases: biochemistry and biology. Annu Rev Cell Dev Biol. 2005; 21:247-269.
22. Zhao Z S, Manser E. PAK and other Rho-associated kinases—effectors with surprisingly diverse mechanisms of regulation. Biochem J. 2005; 386(Pt 2):201-214.
23. Charrasse S, Comunale F, Fortier M, Portales-Casamar E, Debant A, Gauthier-Rouviere C. M-cadherin activates Rac1 GTPase through the Rho-GEF trio during myoblast fusion. Mol Biol Cell. 2007; 18(5): 1734-1743.
24. Castellani L, Salvati E, Alema S, Falcone G. Fine regulation of RhoA and Rock is required for skeletal muscle differentiation. J Biol Chem. 2006; 281(22):15249-15257.
25. Pagiatakis C, Gordon J W, Ehyai S, McDermott J C. A novel RhoA/ROCK-CPI-17-MEF2C signaling pathway regulates vascular smooth muscle cell gene expression. J Biol Chem. 2012; 287(11):8361-8370.
26. Watanabe K, Ueno M, Kamiya D, et al. A ROCK inhibitor permits survival of dissociated human embryonic stem cells. Nat Biotechnol. 2007; 25(6):681-686.
27. Ohgushi M, Matsumura M, Eiraku M, et al. Molecular pathway and cell state responsible for dissociation-induced apoptosis in human pluripotent stem cells. Cell Stem Cell. 2010; 7(2):225-239.
28. Chen G, Hou Z, Gulbranson D R, Thomson J A. Actin-myosin contractility is responsible for the reduced viability of dissociated human embryonic stem cells. Cell Stem Cell. 2010; 7(2):240-248.
29. Laflamme M A, Murry C E. Heart regeneration. Nature. 2011; 473(7347):326-335.
30. Ptaszek L M, Mansour M, Ruskin J N, Chien K R. Towards regenerative therapy for cardiac disease. Lancet. 2012; 379(9819):933-942.
31. Kim Y S, Ahn Y. A long road for stem cells to cure sick hearts: update on recent clinical trials. Korean Circ J. 2012; 42(2):71-79.
32. Kattman S J, Huber T L, Keller G M. Multipotent flk-1+ cardiovascular progenitor cells give rise to the cardiomyocyte, endothelial, and vascular smooth muscle lineages. Dev Cell. 2006; 11(5):723-732.
33. Yang L, Soonpaa M H, Adler E D, et al. Human cardiovascular progenitor cells develop from a KDR+ embryonic-stem-cell-derived population. Nature. 2008; 453(7194):524-528.
34. Burridge P W, Keller G, Gold J D, Wu J C. Production of de novo cardiomyocytes: human pluripotent stem cell differentiation and direct reprogramming. Cell Stem Cell. 2012; 10(1):16-28.
35. Yamashita J K, Takano M, Hiraoka-Kanie M, et al. Prospective identification of cardiac progenitors by a novel single cell-based cardiomyocyte induction. FASEB J. 2005; 19(11):1534-1536.
36. Yan P, Nagasawa A, Uosaki H, et al. Cyclosporin-A potently induces highly cardiogenic progenitors from embryonic stem cells. Biochem Biophys Res Commun. 2009; 379(1):115-120.
37. Fujiwara M, Yan P, Otsuji T G, et al. Induction and enhancement of cardiac cell differentiation from mouse and human induced pluripotent stem cells with cyclosporin-A. PLoS One. 2011; 6(2): e16734.
38. Cho H J, Lee C S, Kwon Y W, et al. Induction of pluripotent stem cells from adult somatic cells by protein-based reprogramming without genetic manipulation. Blood. 2010; 116(3):386-395.
39. Koh Y J, Kim H Z, Hwang S I, et al. Double antiangiogenic protein, DAAP, targeting VEGF-A and angiopoietins in tumor angiogenesis, metastasis, and vascular leakage. Cancer Cell. 2010; 18(2): 171-184.
40. Jeong G S, Han S, Shin Y, et al. Sprouting angiogenesis under a chemical gradient regulated by interactions with an endothelial monolayer in a microfluidic platform. Anal Chem. 2011; 83(22):8454-8459.
41. Chung S, Sudo R, Mack P J, Wan C R, Vickerman V, Kamm R D. Cell migration into scaffolds under co-culture conditions in a microfluidic platform. Lab Chip. 2009; 9(2):269-275.
42. Livak K J, Schmittgen T D. Analysis of relative gene expression data using real-time quantitative PCR and the 2(-Delta Delta C(T)) Method. Methods. 2001; 25(4):402-408.
43. Li Z, Dong X, Wang Z, et al. Regulation of PTEN by Rho small GTPases. Nat Cell Biol. 2005; 7(4):399-404.
44. Fehrenbach M L, Cao G, Williams J T, Finklestein J M, Delisser H M. Isolation of murine lung endothelial cells. Am J Physiol Lung Cell Mol Physiol. 2009; 296(6):L1096-1103.
45. Koh Y J, Koh B I, Kim H, et al. Stromal vascular fraction from adipose tissue forms profound vascular network through the dynamic reassembly of blood endothelial cells. Arterioscler Thromb Vasc Biol. 2011; 31(5):1141-1150.
46. Mack C P, Somlyo A V, Hautmann M, Somlyo A P, Owens G K. Smooth muscle differentiation marker gene expression is regulated by RhoA-mediated actin polymerization. J Biol Chem. 2001; 276(1):341-347.
47. Chen S, Crawford M, Day R M, et al. RhoA modulates Smad signaling during transforming growth factor-beta-induced smooth muscle differentiation. J Biol Chem. 2006; 281(3):1765-1770.
48. Losordo D W, Dimmeler S. Therapeutic angiogenesis and vasculogenesis for ischemic disease: part II: cell-based therapies. Circulation. 2004; 109(22):2692-2697.
49. Dimmeler S, Zeiher A M. Endothelial cell apoptosis in angiogenesis and vessel regression. Circ Res. 2000; 87(6):434-439.
50. Kim I, Ryu Y S, Kwak H J, et al. EphB ligand, ephrinB2, suppresses the VEGF- and angiopoietin 1-induced Ras/mitogen-activated protein kinase pathway in venous endothelial cells. FASEB J. 2002; 16(9):1126-1128.
51. Aitsebaomo J, Portbury A L, Schisler J C, Patterson C. Brothers and sisters: molecular insights into arterial-venous heterogeneity. Circ Res. 2008; 103(9):929-939.
52. Atkins G B, Jain M K, Hamik A. Endothelial differentiation: molecular mechanisms of specification and heterogeneity. Arterioscler Thromb Vasc Biol. 2011; 31(7):1476-1484.
53. Kroll J, Epting D, Kern K, et al. Inhibition of Rho-dependent kinases ROCK I/II activates VEGF-driven retinal neovascularization and sprouting angiogenesis. Am J Physiol Heart Circ Physiol. 2009; 296(3):H893-899.
54. Hoang M V, Whelan M C, Senger D R. Rho activity critically and selectively regulates endothelial cell organization during angiogenesis. Proc Natl Acad Sci USA. 2004; 101(7): 1874-1879.
55. Bryan B A, Dennstedt E, Mitchell D C, et al. RhoA/ROCK signaling is essential for multiple aspects of VEGF-mediated angiogenesis. FASEB J. 2010; 24(9):3186-3195.
56. van Nieuw Amerongen G P, Koolwijk P, Versteilen A, van Hinsbergh V W. Involvement of RhoA/Rho kinase signaling in VEGF-induced endothelial cell migration and angiogenesis in vitro. Arterioscler Thromb Vasc Biol. 2003; 23(2):211-217.
57. McCloskey K E, Smith D A, Jo H, Nerem R M. Embryonic stem cell-derived endothelial cells may lack complete functional maturation in vitro. J Vasc Res. 2006; 43(5):411-421.

All of the references cited herein are incorporated by reference in their entirety.

Those skilled in the art will recognize, or be able to ascertain using no more than routine experimentation, many equivalents to the specific embodiments of the invention specifically described herein.

What is claimed is:

1. A method of promoting differentiation and expansion of endothelial cells (EC) from a pluripotent cell comprising contacting the pluripotent cell with Rho-associated protein kinase (ROCK) suppressor.

2. The method according to claim 1, wherein the ROCK suppressor is RKI, RKI-II, siRNA-ROCK1, si-RNA-ROCK2 or chemical compound specific to ROCK1 or ROCK2.

3. The method according to claim 2, wherein the chemical compound specific to ROCK1 or ROCK2 is Y27632.

4. The method according to claim 1, comprising further contacting the pluripotent cell with VEGF-A.

5. The method according to claim 1, wherein the pluripotent cell is embryonic stem cell (ESC).

6. The method according to claim 1, wherein the pluripotent cell is mesodermal stem cell (MSC).

7. The method according to claim 1, wherein the pluripotent cell is a mammalian cell.

8. The method according to claim 7, wherein the mammalian cell is mouse or human cell.

9. A method of promoting differentiation and expansion of cardiomyocytes (CM) from a pluripotent cell comprising contacting the pluripotent cell with Rho-associated protein kinase (ROCK) suppressor.

10. The method according to claim 9, wherein the ROCK suppressor is RKI, RKI-II, siRNA-ROCK1, si-RNA-ROCK2 or chemical compound specific to ROCK1 or ROCK2.

11. The method according to claim 10, wherein the chemical compound specific to ROCK1 or ROCK2 is Y27632.

12. The method according to claim 9, comprising further contacting the pluripotent cell with an immunosuppressant.

13. The method according to claim 12, wherein the immunosuppressant is Cyclosporine A.

14. The method according to claim 9, wherein the pluripotent cell is embryonic stem cell (ESC).

15. The method according to claim 9, wherein the pluripotent cell is mesodermal stem cell (MSC).

16. The method according to claim 9, wherein the pluripotent cell is a mammalian cell.

17. The method according to claim 16, wherein the mammalian cell is mouse or human cell.

18. A method of creating new blood vessels comprising contacting pluripotent cell with Rho-associated protein kinase (ROCK) suppressor.

19. The method according to claim 18, wherein the ROCK suppressor is RKI, RKI-II, siRNA-ROCK1, si-RNA-ROCK2 or chemical compound specific to ROCK1 or ROCK2.

20. The method according to claim 19, wherein the chemical compound specific to ROCK1 or ROCK2 is Y27632.
